(19) United States
(12) Patent Application Publication
(10) Pub. No.: US 2021/0275522 A1
(43) Pub. Date: Sep. 9, 2021

(54) PHARMACEUTICAL COMPOSITIONS AND USE THEREOF

(71) Applicants: ASCENTAGE PHARMA (SUZHOU) CO., LTD., Suzhou (CN); ASCENTAGE PHARMA GROUP CORP LIMITED, Hong Kong (CN)

(72) Inventors: Dajun YANG, Suzhou (CN); Yifan ZHAI, Suzhou (CN); Douglas Dong FANG, Suzhou (CN); Ran TAO, Suzhou (CN)

(21) Appl. No.: 16/966,736

(22) PCT Filed: Nov. 22, 2019
(86) PCT No.: PCT/CN2019/120144

§ 371 Date: Jul. 31, 2020

(30) Foreign Application Priority Data
Nov. 23, 2018 (WO) PCTCN2018117270
Nov. 19, 2019 (CN) 201911132493.3

(51) Int. Cl. A61K 31/496; A61K 31/437; A61K 31/453; A61K 31/519; A61K 45/06; A61P 35/04

(52) U.S. Cl. CPC A61K 31/496; A61K 31/437; A61P 35/04; A61K 31/519; A61K 45/06; A61K 31/453

(57) ABSTRACT

Disclosed is a pharmaceutical composition comprising (i) a compound of formula (I) or a pharmaceutically acceptable salt thereof; (ii) a CDK inhibitor or a pharmaceutically acceptable salt thereof, for the prevention and/or treatment of a disease mediated by Bcl-2 and/or CDK activity.

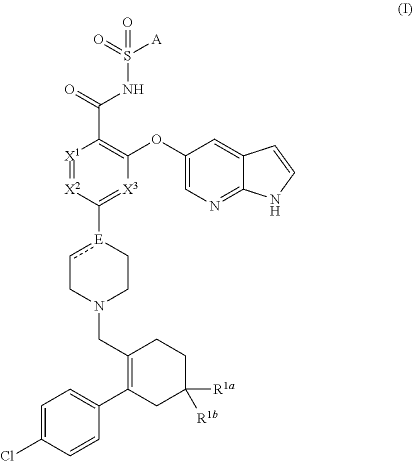

The present application claims the benefits of PCT/CN2018/117270 filed on Nov. 23, 2018 and the Chinese Patent Application No. CN201911132493.3 filed on Nov. 19, 2019, the contents of which are incorporated herein by reference in their entireties.

### FIELD OF THE INVENTION

The present invention relates to a pharmaceutical composition and a use thereof.

### BACKGROUND OF THE INVENTION

Pharmacologic approaches for treating cancer have traditionally relied on the use of various single agent systemic therapies. But evidence is mounting that tumors may not be complete responsive to monotherapies. Effective treatments are urgently needed for treating cancer, such as AML (acute myeloid leukemia) and relapsed or refractory DLBCL (diffuse large B-cell lymphoma). Elderly AML and relapsed or refractory DLBCL patients as only 10-25% of the population respond to standard therapies and resistance often occurred. Concurrent overexpression of anti-apoptotic proteins BCL-2 and MYC are found in AML and DLBCL patients, while recent studies suggested that this molecular characteristic is associated with poor prognosis in DLBLC and AML (Hu S, Xu-Monette Z Y, Tzankov A, et al., Blood, 2013, 121, 4021-31; Thol F, Schlenk R F, Heuser M, Ganser A, Blood, 2015, 126, 319-327; Breems D A, Van Putten W L, Huijgens P C, Ossenkoppele G J, Verhoef G E G, Verdonck L F, et al., J Clin Oncol, 2005, 23, 1969-1978).

CDK9 controls non-ribosomal transcription of genes including proto-oncogene MYC and anti-apoptotic gene MCL-1. Dysregulation in the CDK9 pathway had been reported in various hematologic malignancies, making it an attractive target for novel therapies and combinations (Boffo S, Damato A, Alfano L, et al., Journal of Experimental & Clinical Cancer Research, 2018, 37(1): 36).

Bcl-2 is the founding member of the Bcl-2 family of regulator proteins that regulate cell death (apoptosis), by either inducing (pro-apoptotic) or inhibiting (anti-apoptotic) apoptosis.

### CONTENT OF THE INVENTION

In one aspect, the present invention provides a pharmaceutical composition comprising

(i) a compound of formula (I) or a pharmaceutically acceptable salt thereof, and,

(ii) a CDK inhibitor or a pharmaceutically acceptable salt thereof;

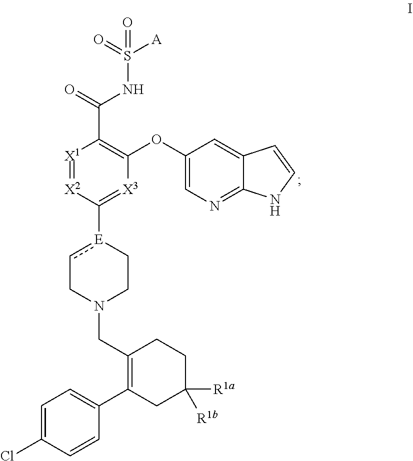

wherein, A is selected from the group consisting of

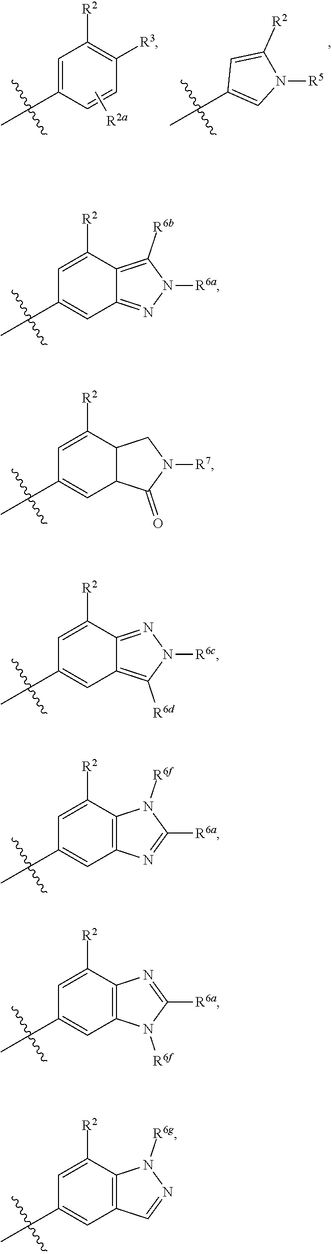

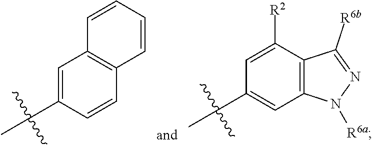

E is a carbon atom and [symbol] is a double bond; or

E is a —C(H)— and [symbol] is a single bond, or

E is a nitrogen atom and [symbol] is a single bond;

X¹, X², and X³ are each independently selected from the group consisting of —CR⁸═ and —N═;

R^{1a} and R^{1b} taken together with the carbon atom to which they are attached form a 3-, 4-, or 5-membered optionally substituted cycloalkyl; or

R^{1a} and R^{1b} taken together with the carbon atom to which they are attached form a 4- or 5-membered optionally substituted heterocyclo;

each of R² is independently selected from the group consisting of —NO₂, —SO₂CH₃, and —SO₂CF₃;

each of R^{2a} is independently selected from the group consisting of hydrogen and halogen;

R³ is selected from the group consisting of hydrogen, —CN, —C≡CH, and —N(R^{4a})(R^{4b});

R^{4a} is selected from the group consisting of optionally substituted C₁₋₆ alkyl, optionally substituted C₃₋₆ cycloalkyl, heterocyclo, heteroalkyl, (cycloalkyl)alkyl, and (heterocyclo)alkyl;

R^{4b} is selected from the group consisting of hydrogen and C₁₋₄ alkyl;

R⁵ is selected from the group consisting of optionally substituted C₁₋₆ alkyl, heterocyclo, heteroalkyl, (cycloalkyl)alkyl, and (heterocyclo)alkyl;

R^{6a}, R^{6c}, R^{6e}, R^{6f}, and R^{6g} are each independently selected from the group consisting of hydrogen, optionally substituted C₁₋₆ alkyl, optionally substituted C₃₋₆ cycloalkyl, optionally substituted aryl, optionally substituted heteroaryl, heterocyclo, heteroalkyl, (cycloalkyl)alkyl, and (heterocyclo)alkyl;

R^{6b} and R^{6d} are each independently selected from the group consisting of hydrogen, C₁₋₄ alkyl, and halogen;

R⁷ is selected from the group consisting of optionally substituted C₁₋₆ alkyl, heterocyclo, heteroalkyl, (cycloalkyl)alkyl, and (heterocyclo)alkyl; and

R⁸ is selected from the group consisting of hydrogen and halogen.

In some embodiments, the compound of formula (I) is selected from the group consisting of

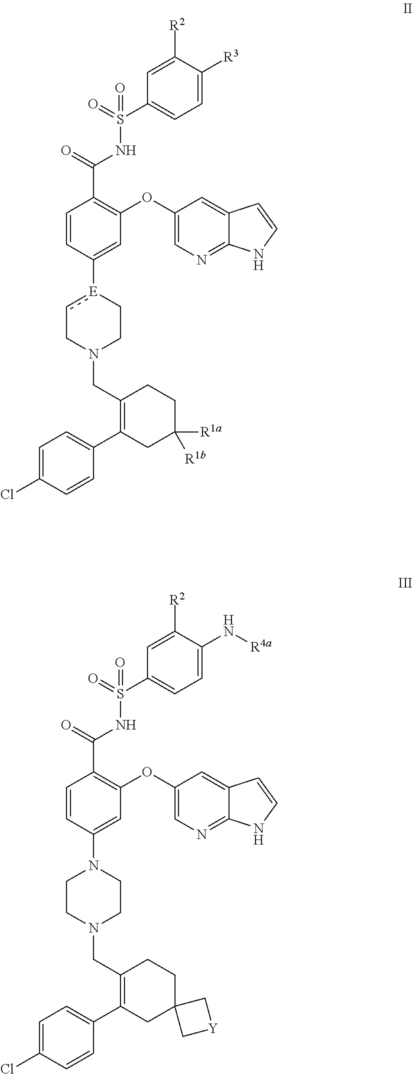

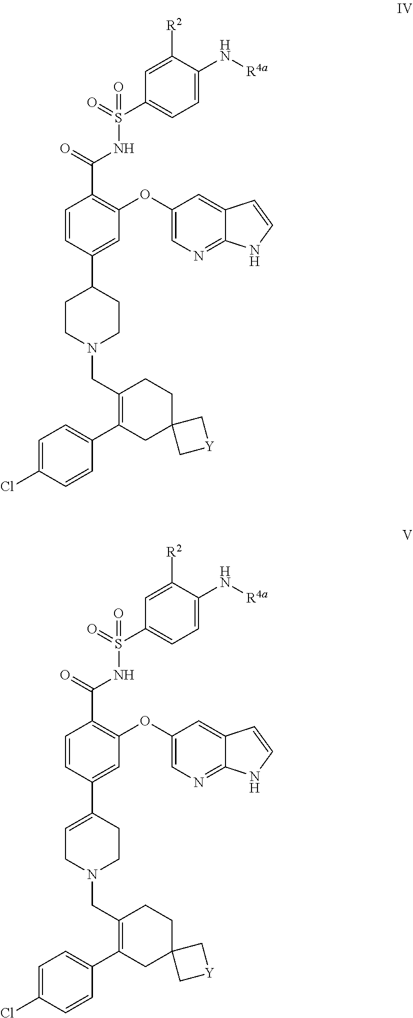

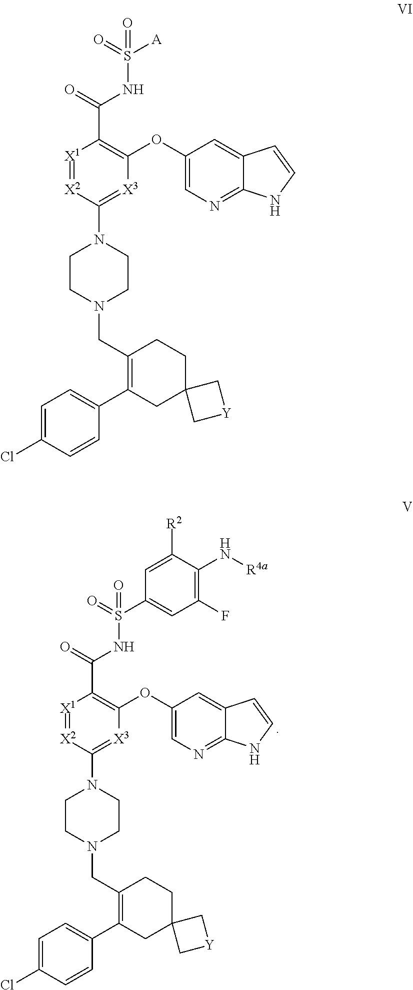

In some embodiments, R^{4a} is selected from the group consisting of

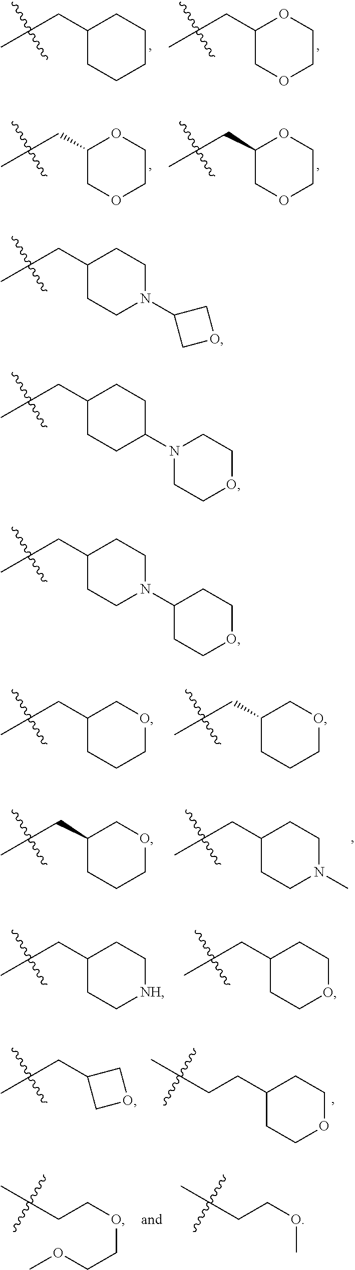

In some embodiments, the compound of formula (I) is selected from

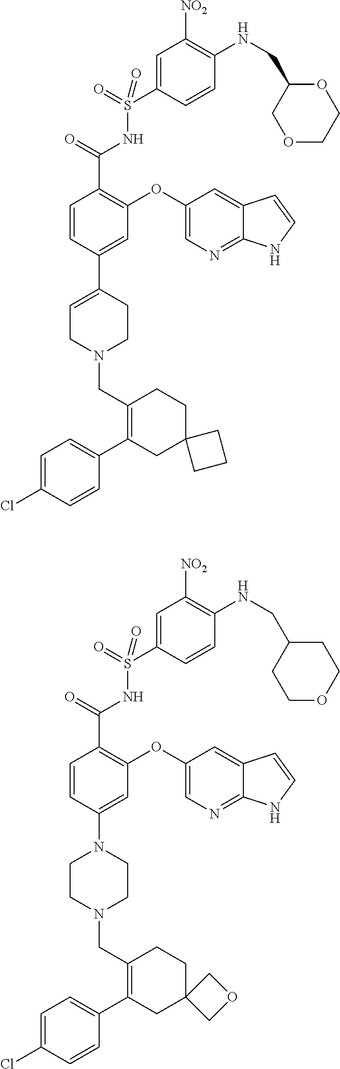 [SMILES: O=C(NS(=O)(=O)c1ccc(NC[C@@H]2COCCO2)c([N+](=O)[O-])c1)c1ccc(C2=CCN(CC3=C(c4ccc(Cl)cc4)CC4(CCC4)CC3)CC2)cc1Oc1cnc2[nH]ccc2c1 ; O=C(NS(=O)(=O)c1ccc(NCC2CCOCC2)c([N+](=O)[O-])c1)c1ccc(N2CCN(CC3=C(c4ccc(Cl)cc4)CC4(CC3)COC4)CC2)cc1Oc1cnc2[nH]ccc2c1]

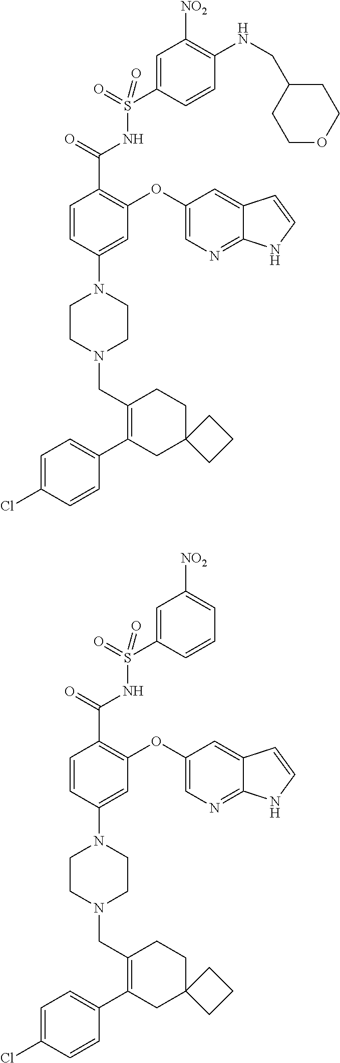 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1cccc([N+](=O)[O-])c1)c1ccc(N2CCN(CC3=C(c4ccc(Cl)cc4)CC4(CCC4)CC3)CC2)cc1Oc1cnc2[nH]ccc2c1]

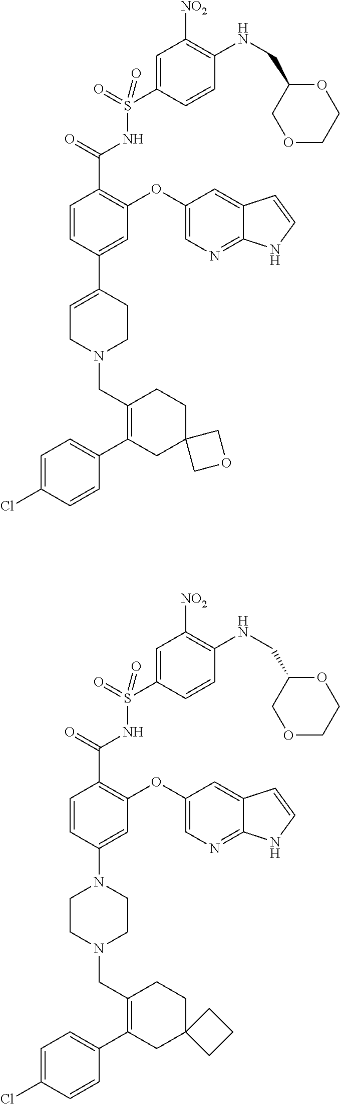 [SMILES: O=C(NS(=O)(=O)c1ccc(NC[C@@H]2COCCO2)c([N+](=O)[O-])c1)c1ccc(C2=CCN(CC3=C(c4ccc(Cl)cc4)CC4(CC3)COC4)CC2)cc1Oc1cnc2[nH]ccc2c1 ; O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

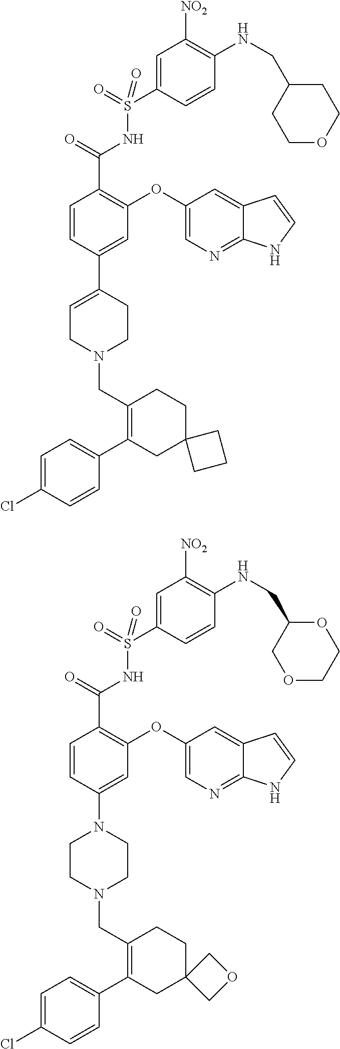 [SMILES: O=C(NS(=O)(=O)c1ccc(NCC2CCOCC2)c([N+](=O)[O-])c1)c1ccc(C2=CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@@H]2COCCO2)c([N+](=O)[O-])c1)c1ccc(N2CCN(CC3=C(c4ccc(Cl)cc4)CC4(CC3)COC4)CC2)cc1Oc1cnc2[nH]ccc2c1]

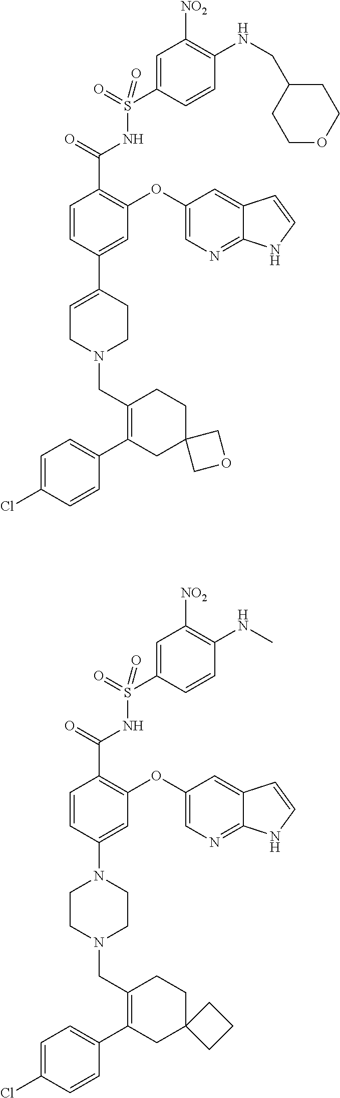 [SMILES: O=C(NS(=O)(=O)c1ccc(NCC2CCOCC2)c([N+](=O)[O-])c1)c1ccc(C2=CCN(CC3=C(c4ccc(Cl)cc4)CC4(CC3)COC4)CC2)cc1Oc1cnc2[nH]ccc2c1 ; CNc1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]]

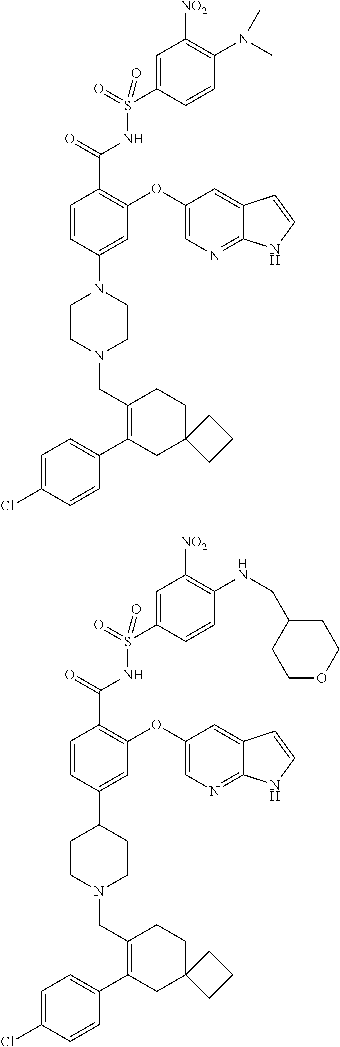 [SMILES: CN(C)c1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-] ; O=C(NS(=O)(=O)c1ccc(NCC2CCOCC2)c([N+](=O)[O-])c1)c1ccc(C2CCN(CC3=C(c4ccc(Cl)cc4)CC4(CCC4)CC3)CC2)cc1Oc1cnc2[nH]ccc2c1]

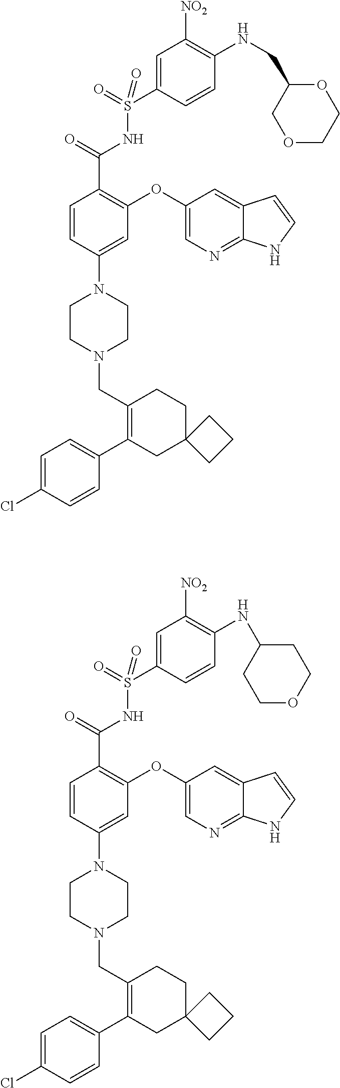 [SMILES: O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC2CCOCC2)c([N+](=O)[O-])c1)c1ccc(N2CCN(CC3=C(c4ccc(Cl)cc4)CC4(CCC4)CC3)CC2)cc1Oc1cnc2[nH]ccc2c1]

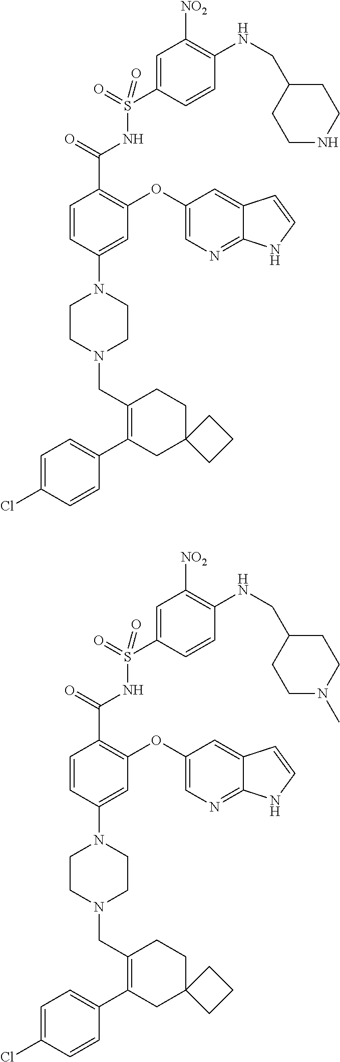 [SMILES: O=C(NS(=O)(=O)c1ccc(NCC2CCNCC2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; CN1CCC(CNc2ccc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc2[N+](=O)[O-])CC1]

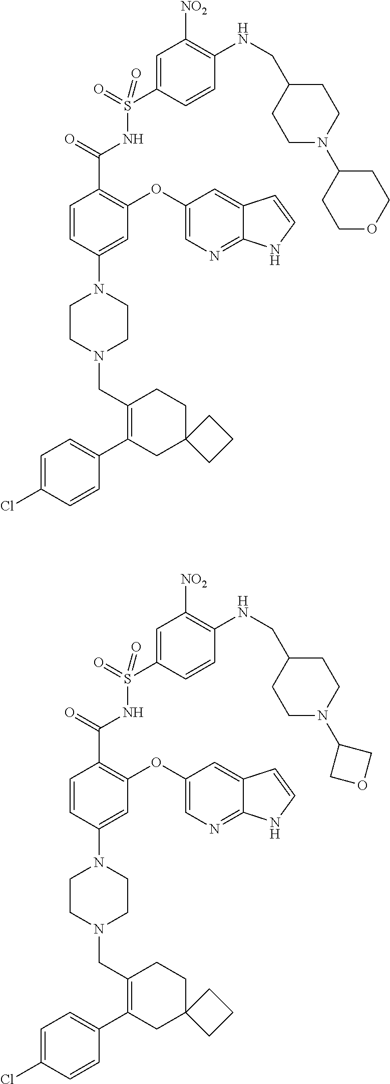 [SMILES: O=C(NS(=O)(=O)c1ccc(NCC2CCN(C3CCOCC3)CC2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NCC2CCN(C3COC3)CC2)c([N+](=O)[O-])c1)c1ccc(N2CCN(CC3=C(c4ccc(Cl)cc4)CC4(CCC4)CC3)CC2)cc1Oc1cnc2[nH]ccc2c1]

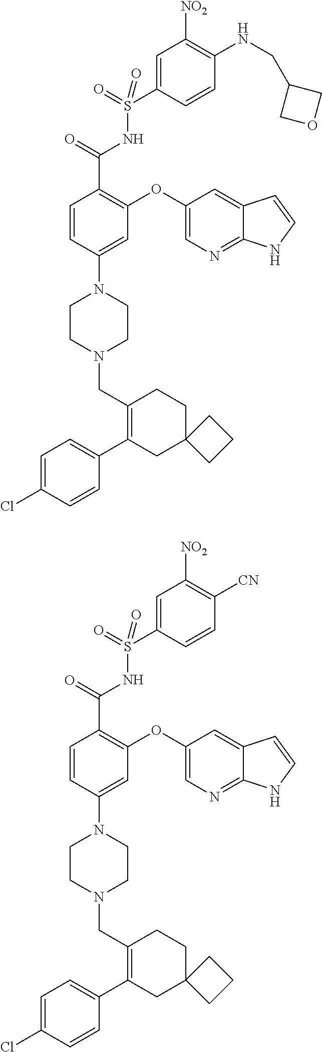 [SMILES: O=C(NS(=O)(=O)c1ccc(NCC2COC2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; N#Cc1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]]

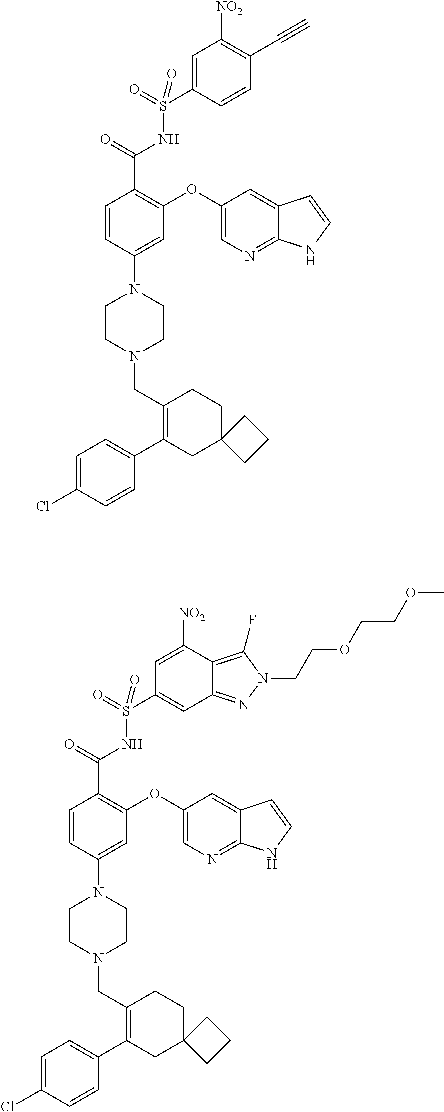 [SMILES: C#Cc1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-] ; COCCOCCn1nc2cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc([N+](=O)[O-])c2c1F]

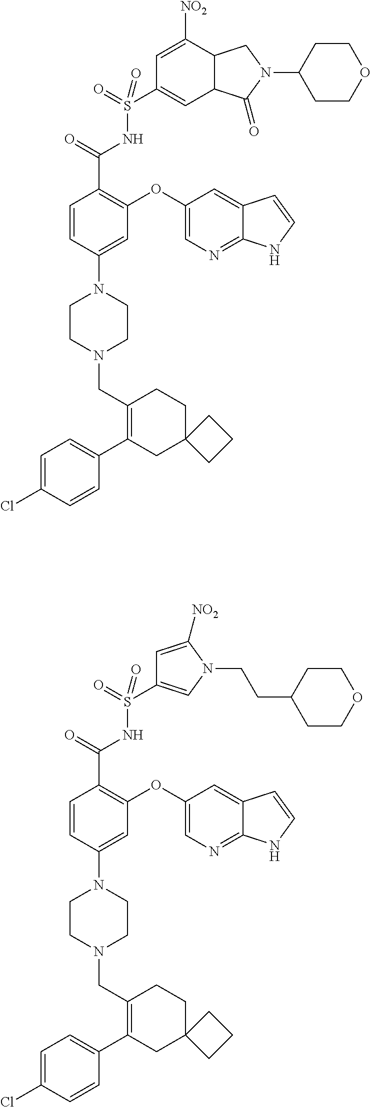 [SMILES: O=C(NS(=O)(=O)C1=CC2C(=O)N(C3CCOCC3)CC2C([N+](=O)[O-])=C1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1cc([N+](=O)[O-])n(CCC2CCOCC2)c1)c1ccc(N2CCN(CC3=C(c4ccc(Cl)cc4)CC4(CCC4)CC3)CC2)cc1Oc1cnc2[nH]ccc2c1]

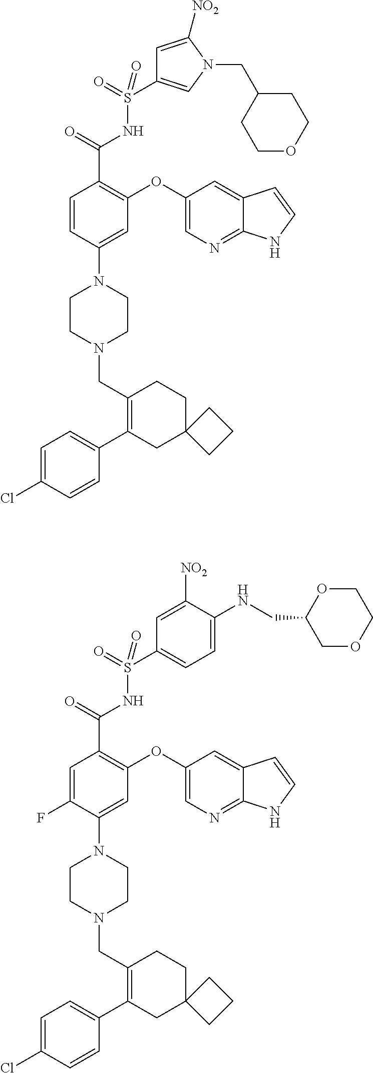 [SMILES: O=C(NS(=O)(=O)c1cc([N+](=O)[O-])n(CC2CCOCC2)c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@H]2COCCO2)c([N+](=O)[O-])c1)c1cc(F)c(N2CCN(CC3=C(c4ccc(Cl)cc4)CC4(CCC4)CC3)CC2)cc1Oc1cnc2[nH]ccc2c1]

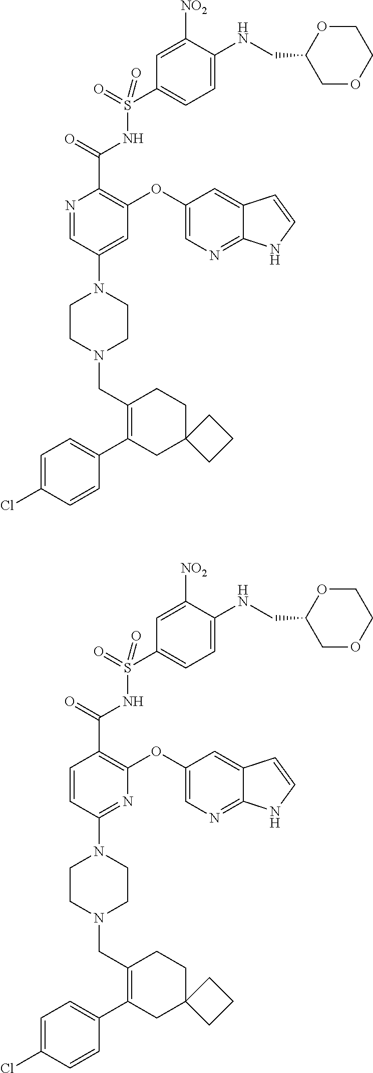 [SMILES: O=C(NS(=O)(=O)c1ccc(NC[C@H]2COCCO2)c([N+](=O)[O-])c1)c1ncc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)nc1Oc1cnc2[nH]ccc2c1]

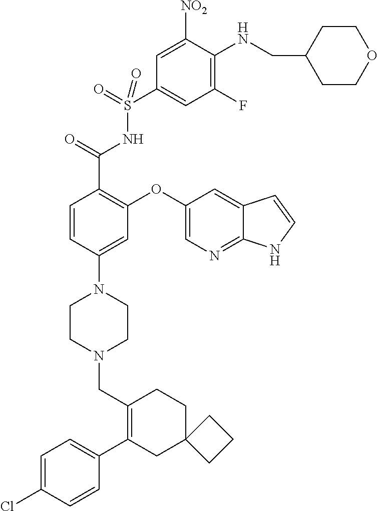 [SMILES: O=C(NS(=O)(=O)c1cc(F)c(NCC2CCOCC2)c([N+](=O)[O-])c1)c1ccc(N2CCN(CC3=C(c4ccc(Cl)cc4)CC4(CCC4)CC3)CC2)cc1Oc1cnc2[nH]ccc2c1]

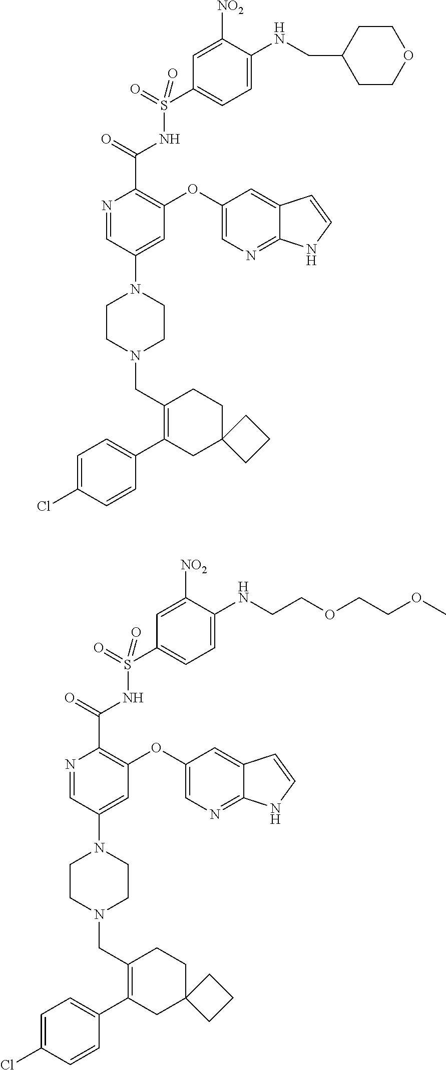 [SMILES: O=C(NS(=O)(=O)c1ccc(NCC2CCOCC2)c([N+](=O)[O-])c1)c1ncc(N2CCN(CC3=C(c4ccc(Cl)cc4)CC4(CCC4)CC3)CC2)cc1Oc1cnc2[nH]ccc2c1 ; COCCOCCNc1ccc(S(=O)(=O)NC(=O)c2ncc(N3CCN(CC4=C(c5ccc(Cl)cc5)CC5(CCC5)CC4)CC3)cc2Oc2cnc3[nH]ccc3c2)cc1[N+](=O)[O-]]

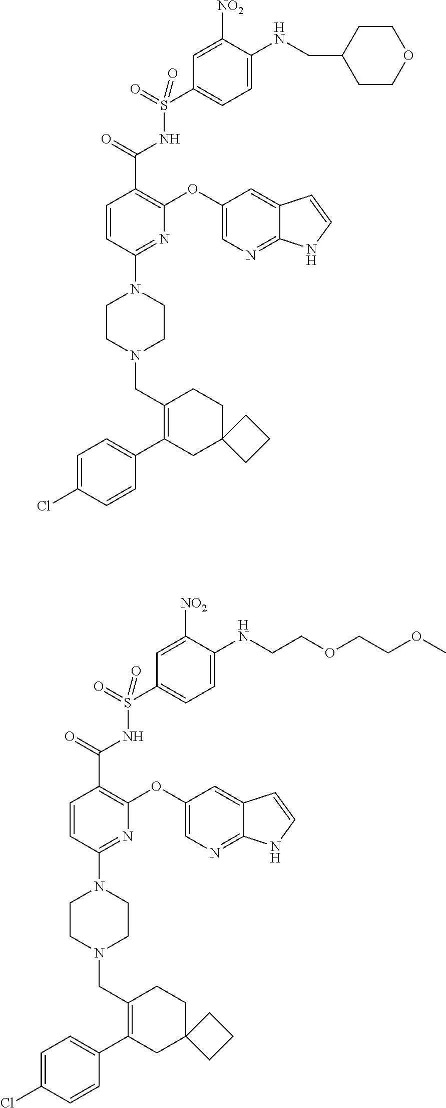 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CCC4)CC3)CC2)nc1Oc1cnc2[nH]ccc2c1 ; COCCOCCNc1ccc(S(=O)(=O)NC(=O)c2ccc(N3CCN(CC4=C(c5ccc(Cl)cc5)CC5(CCC5)CC4)CC3)nc2Oc2cnc3[nH]ccc3c2)cc1[N+](=O)[O-]]

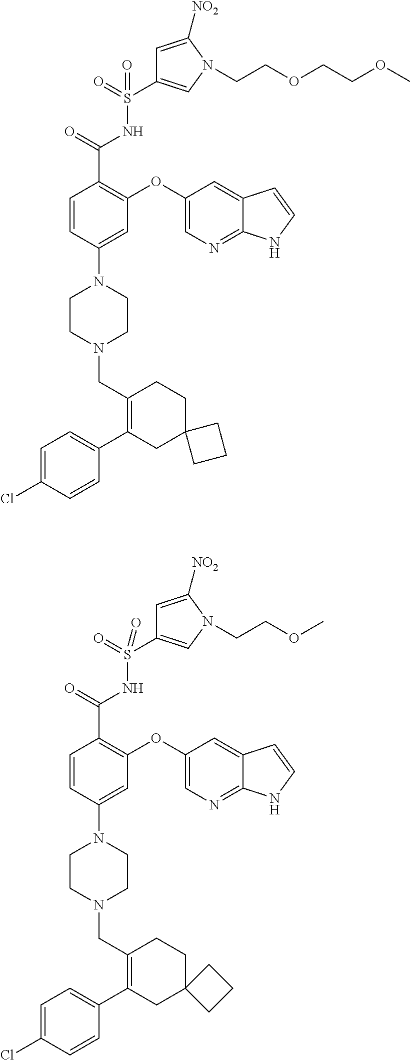 [SMILES: COCCOCCn1cc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-] ; COCCn1cc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]]

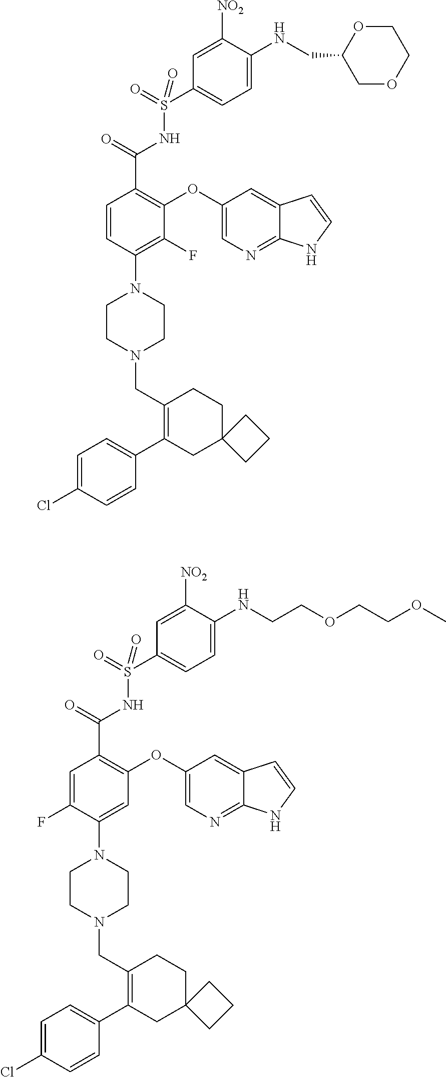 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)c(F)c1Oc1cnc2[nH]ccc2c1 ; COCCOCCNc1ccc(S(=O)(=O)NC(=O)c2cc(F)c(N3CCN(CC4=C(c5ccc(Cl)cc5)CC5(CCC5)CC4)CC3)cc2Oc2cnc3[nH]ccc3c2)cc1[N+](=O)[O-]]

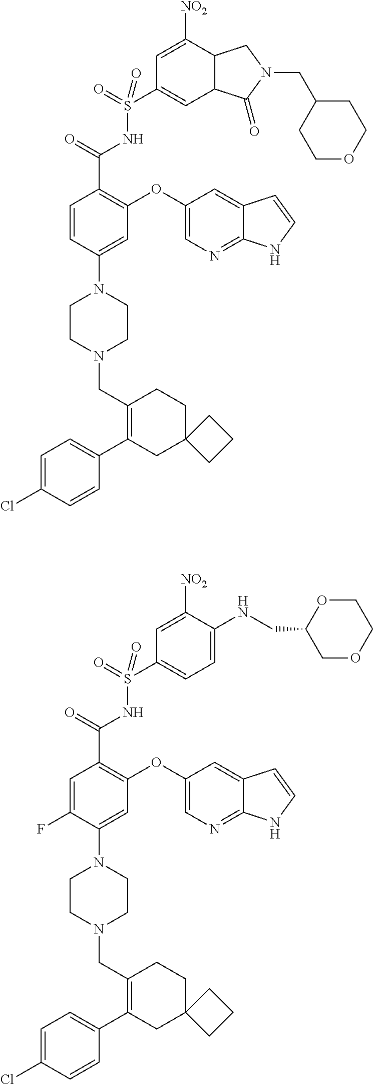 [SMILES: O=C(NS(=O)(=O)C1=CC2C(=O)N(CC3CCOCC3)CC2C([N+](=O)[O-])=C1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@H]2COCCO2)c([N+](=O)[O-])c1)c1cc(F)c(N2CCN(CC3=C(c4ccc(Cl)cc4)CC4(CCC4)CC3)CC2)cc1Oc1cnc2[nH]ccc2c1]

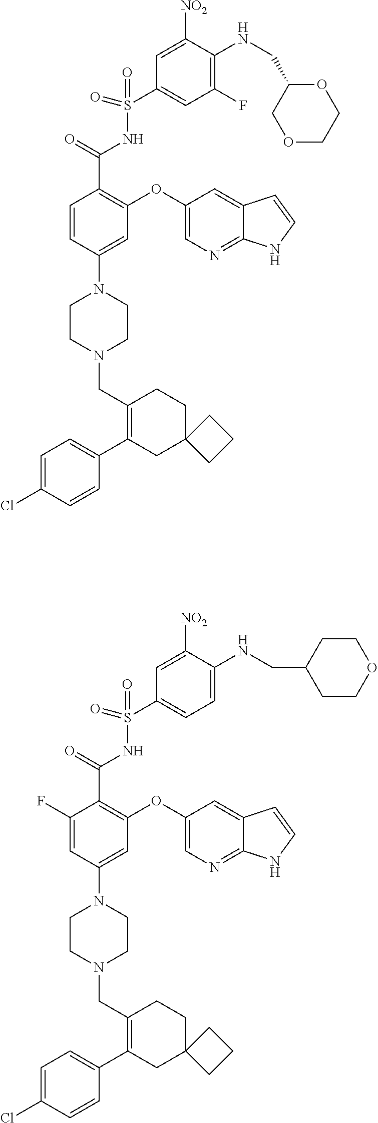 [SMILES: O=C(NS(=O)(=O)c1cc(F)c(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NCC2CCOCC2)c([N+](=O)[O-])c1)c1c(F)cc(N2CCN(CC3=C(c4ccc(Cl)cc4)CC4(CCC4)CC3)CC2)cc1Oc1cnc2[nH]ccc2c1]

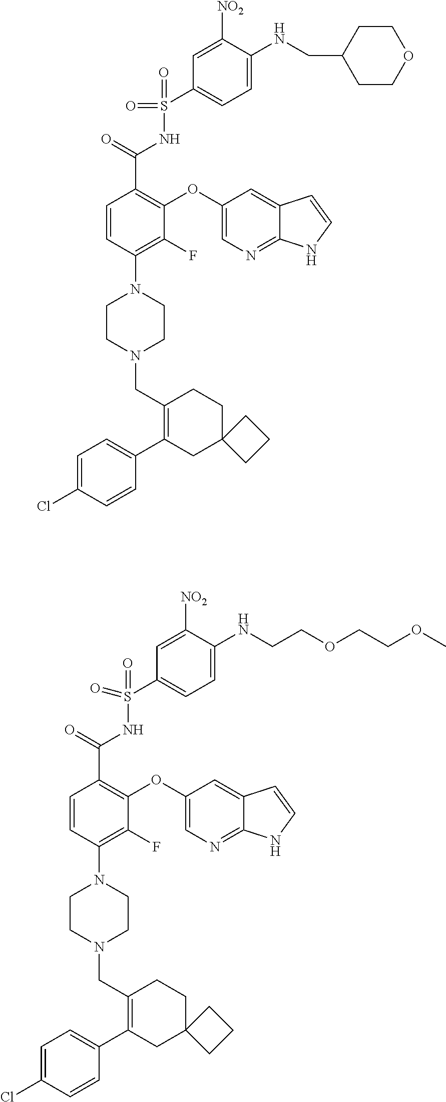 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CCC4)CC3)CC2)c(F)c1Oc1cnc2[nH]ccc2c1 ; COCCOCCNc1ccc(S(=O)(=O)NC(=O)c2ccc(N3CCN(CC4=C(c5ccc(Cl)cc5)CC5(CCC5)CC4)CC3)c(F)c2Oc2cnc3[nH]ccc3c2)cc1[N+](=O)[O-]]

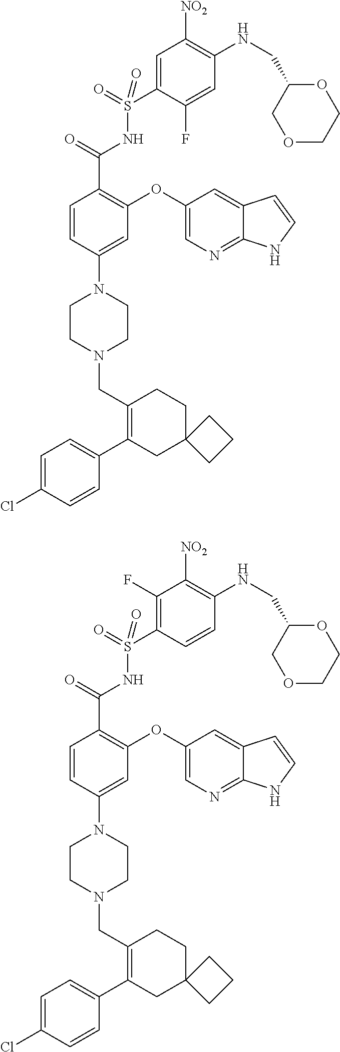 [SMILES: O=C(NS(=O)(=O)c1cc([N+](=O)[O-])c(NC[C@H]2COCCO2)cc1F)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@H]2COCCO2)c([N+](=O)[O-])c1F)c1ccc(N2CCN(CC3=C(c4ccc(Cl)cc4)CC4(CCC4)CC3)CC2)cc1Oc1cnc2[nH]ccc2c1]

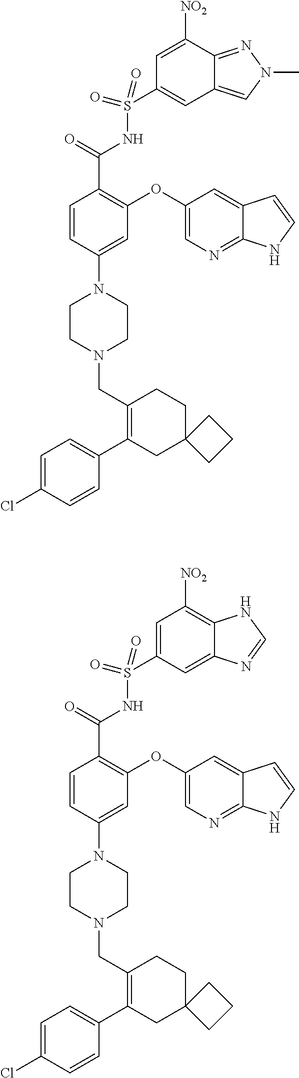 [SMILES: Cn1cc2cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc([N+](=O)[O-])c2n1 ; O=C(NS(=O)(=O)c1cc([N+](=O)[O-])c2[nH]cnc2c1)c1ccc(N2CCN(CC3=C(c4ccc(Cl)cc4)CC4(CCC4)CC3)CC2)cc1Oc1cnc2[nH]ccc2c1]

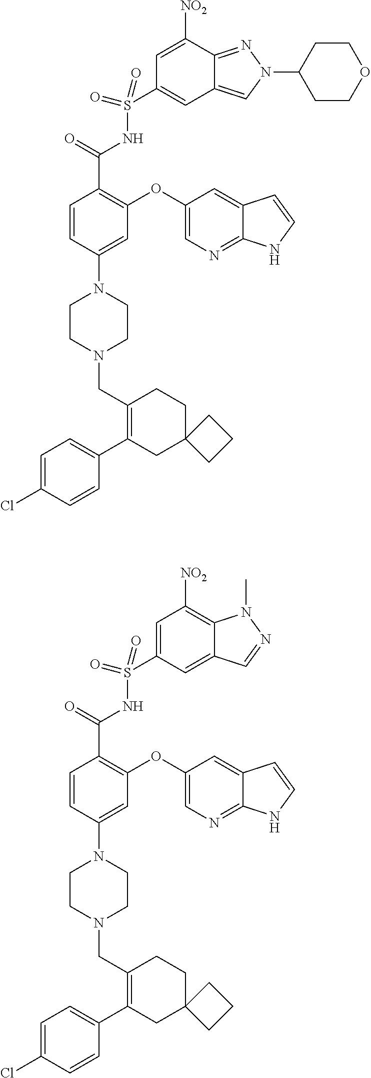 [SMILES: O=C(NS(=O)(=O)c1cc([N+](=O)[O-])c2nn(C3CCOCC3)cc2c1)c1ccc(N2CCN(CC3=C(c4ccc(Cl)cc4)CC4(CCC4)CC3)CC2)cc1Oc1cnc2[nH]ccc2c1 ; Cn1ncc2cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc([N+](=O)[O-])c21]

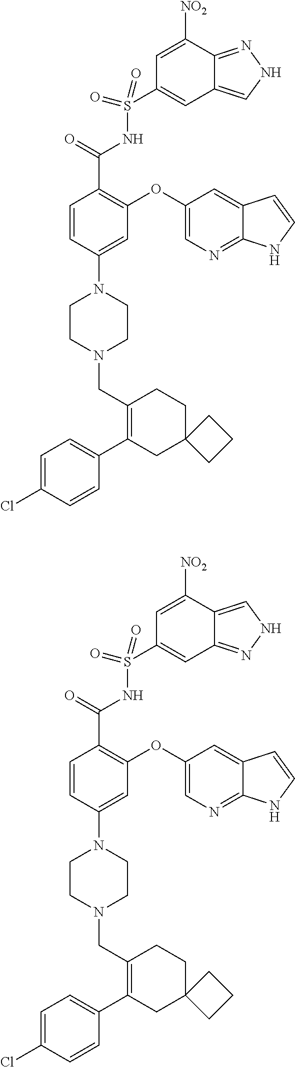 [SMILES: O=C(NS(=O)(=O)c1cc([N+](=O)[O-])c2n[nH]cc2c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1cc([N+](=O)[O-])c2c[nH]nc2c1)c1ccc(N2CCN(CC3=C(c4ccc(Cl)cc4)CC4(CCC4)CC3)CC2)cc1Oc1cnc2[nH]ccc2c1]

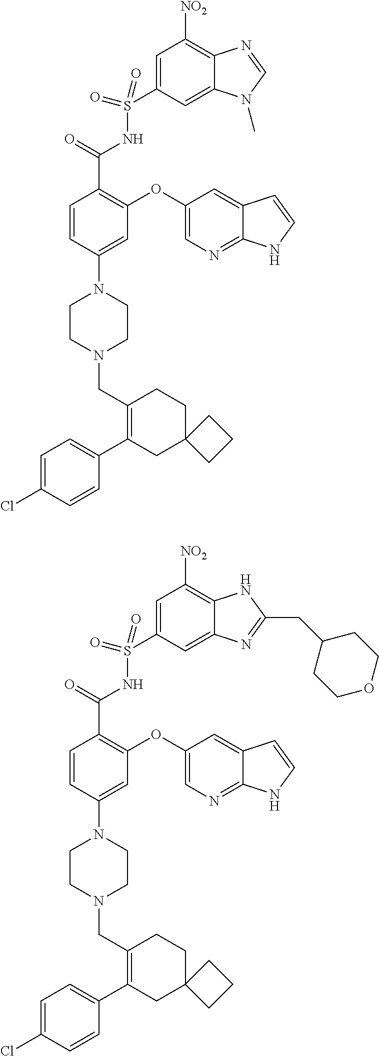 [SMILES: Cn1cnc2c([N+](=O)[O-])cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc21 ; O=C(NS(=O)(=O)c1cc([N+](=O)[O-])c2[nH]c(CC3CCOCC3)nc2c1)c1ccc(N2CCN(CC3=C(c4ccc(Cl)cc4)CC4(CCC4)CC3)CC2)cc1Oc1cnc2[nH]ccc2c1]

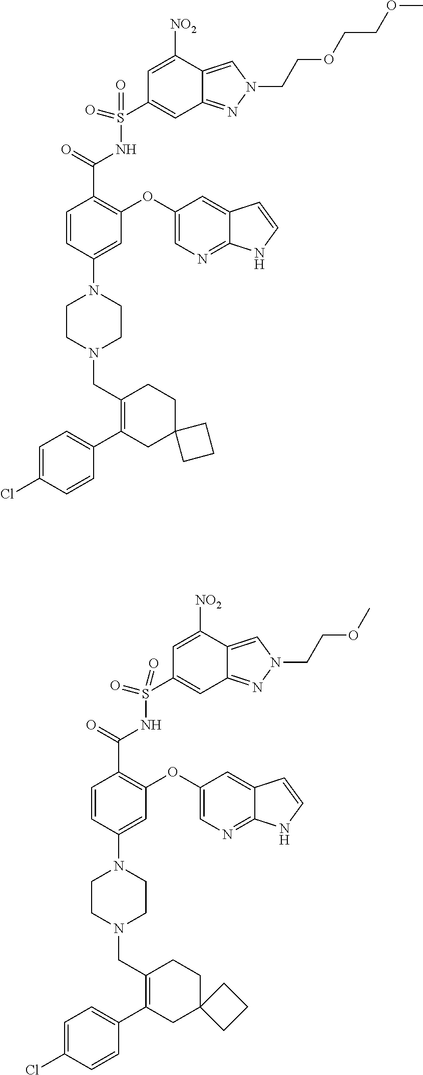 [SMILES: COCCOCCn1cc2c([N+](=O)[O-])cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc2n1 ; COCCn1cc2c([N+](=O)[O-])cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc2n1]

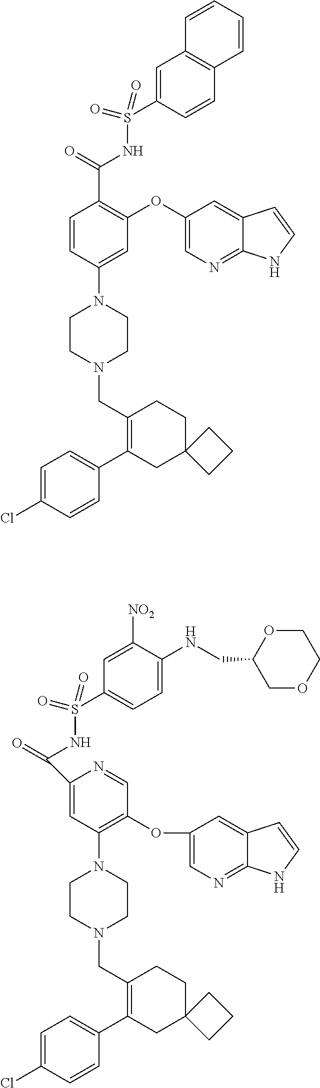 [SMILES: O=C(NS(=O)(=O)c1ccc2ccccc2c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@H]2COCCO2)c([N+](=O)[O-])c1)c1cc(N2CCN(CC3=C(c4ccc(Cl)cc4)CC4(CCC4)CC3)CC2)c(Oc2cnc3[nH]ccc3c2)cn1]

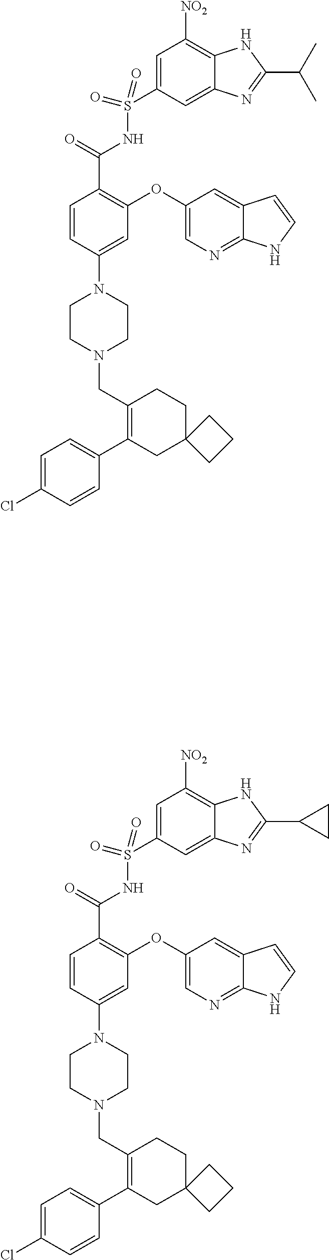 [SMILES: CC(C)c1nc2cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc([N+](=O)[O-])c2[nH]1 ; O=C(NS(=O)(=O)c1cc([N+](=O)[O-])c2[nH]c(C3CC3)nc2c1)c1ccc(N2CCN(CC3=C(c4ccc(Cl)cc4)CC4(CCC4)CC3)CC2)cc1Oc1cnc2[nH]ccc2c1]

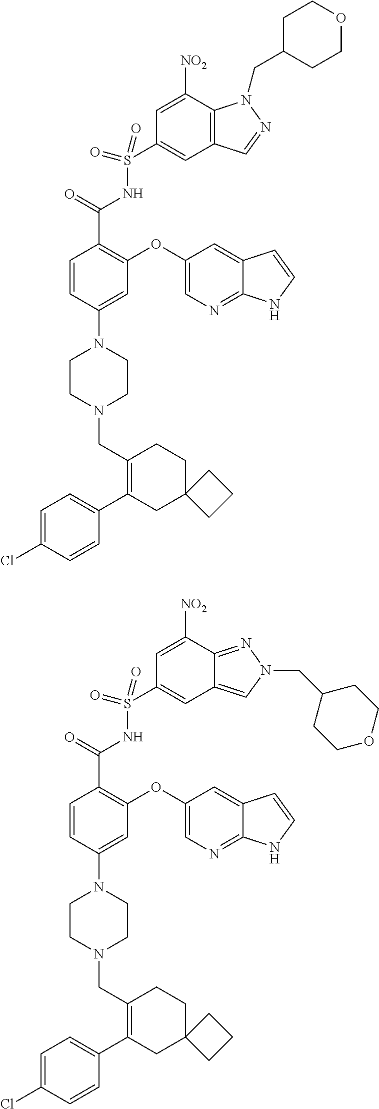 [SMILES: O=C(NS(=O)(=O)c1cc([N+](=O)[O-])c2c(cnn2CC2CCOCC2)c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1cc([N+](=O)[O-])c2nn(CC3CCOCC3)cc2c1)c1ccc(N2CCN(CC3=C(c4ccc(Cl)cc4)CC4(CCC4)CC3)CC2)cc1Oc1cnc2[nH]ccc2c1]

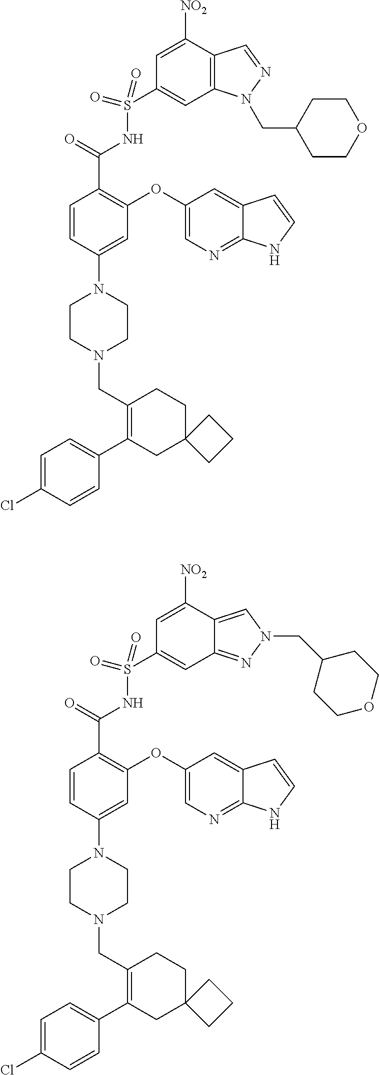 [SMILES: O=C(NS(=O)(=O)c1cc([N+](=O)[O-])c2cnn(CC3CCOCC3)c2c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1cc([N+](=O)[O-])c2cn(CC3CCOCC3)nc2c1)c1ccc(N2CCN(CC3=C(c4ccc(Cl)cc4)CC4(CCC4)CC3)CC2)cc1Oc1cnc2[nH]ccc2c1]

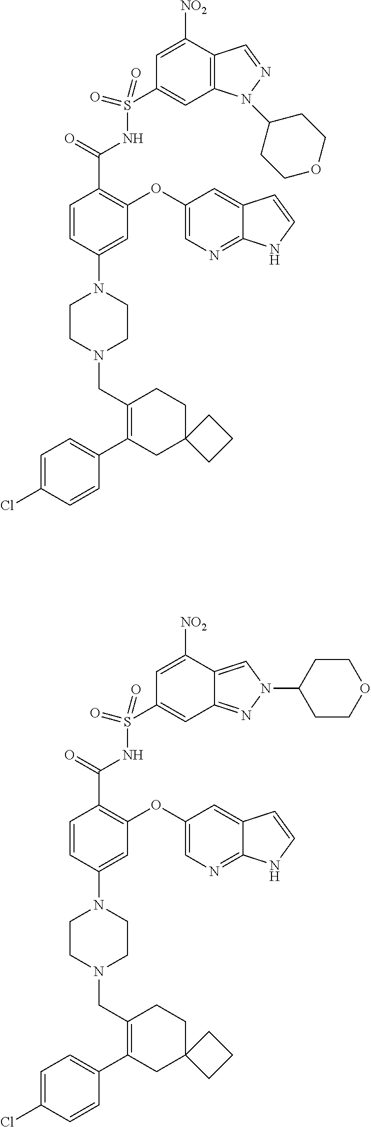 [SMILES: O=C(NS(=O)(=O)c1cc([N+](=O)[O-])c2cnn(C3CCOCC3)c2c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1cc([N+](=O)[O-])c2cn(C3CCOCC3)nc2c1)c1ccc(N2CCN(CC3=C(c4ccc(Cl)cc4)CC4(CCC4)CC3)CC2)cc1Oc1cnc2[nH]ccc2c1]

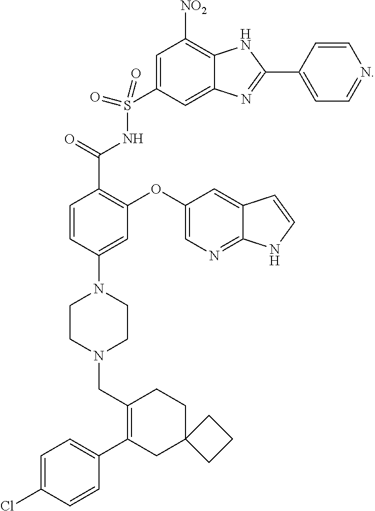 [SMILES: O=C(NS(=O)(=O)c1cc([N+](=O)[O-])c2[nH]c(-c3ccncc3)nc2c1)c1ccc(N2CCN(CC3=C(c4ccc(Cl)cc4)CC4(CCC4)CC3)CC2)cc1Oc1cnc2[nH]ccc2c1]

In another aspect, the present invention provides a pharmaceutical combination comprising

(i) a compound of formula (I) or a pharmaceutically acceptable salt thereof; and,

(ii) a CDK inhibitor or a pharmaceutically acceptable salt thereof.

In the above composition or combination, component (i) (i.e. the compound of formula (I)) and component (ii) (i.e. the CDK inhibitor) can be present in one combined unit dosage form or in two or more separate unit dosage forms. The unit dosage form may also be a fixed combination.

The present invention also provides a kit comprising in separate containers in a single package pharmaceutical compositions comprising in one container a pharmaceutical composition comprising a compound of formula (I) or a pharmaceutically acceptable salt thereof, and in a second container a pharmaceutical composition comprising a CDK inhibitor or a pharmaceutically acceptable salt thereof. The kit form is particularly advantageous when the separate components must be administered in different dosage forms (e.g. oral compound of formula (I) formulation and parenteral CDK inhibitor formulation) or are administered at different dosage intervals.

In another aspect, the present invention provides a use of the pharmaceutical composition or combination according to the present invention in manufacturing a medicament for the prevention and/or treatment of a disease mediated by Bel-2 and/or CDK activity.

In another aspect, the present invention provides a method for the prevention and/or treatment of a disease mediated by Bel-2 and/or CDK activity, which comprises administering to a subject in need thereof a therapeutically effective amount of the pharmaceutical composition or combination according to the present invention. Each of the component of the composition or combination according to the present invention may be administered simultaneously or separately in any order.

In another aspect, the present invention provides a method for the prevention and/or treatment of a disease mediated by Bcl-2 and/or CDK activity, which comprises administering to a subject in need thereof a therapeutically effective amount of a compound of formula (I), and a therapeutically effective amount of a CDK inhibitor. The compound of formula (I) and the CDK inhibitor may be administered simultaneously or separately in any order.

The CDK inhibitor can be selected from the group consisting of kenpaullone, PKC-412, butyrolactone I, alvocidib, N9-isopropyl-olomoucine, indirubin-3′-monoxime, NU2058, olomoucine II, 9-cyanopaullone, 5-iodo-indirubin-3′-monoxime, NU6102, oxindole I, SU 9516, roscovitine, RO-3306, 10Z-hymenialdisine, AZD 5438, AT7519, dinaciclib, R547, CGP 74514A, SNS-032 (BMS-387032), BMS-265246, JNJ-7706621, PHA-793887, P276-00, PHA-767491, milciclib (PHA-848125), NU6027, LDC000067, ribociclib (Kisqali®), palbociclib (Ibrance®), abemaciclib (Vernenio®), Senexin A, Atuveciclib, LY2857785, and dinaciclib.

In some embodiments, the compound of formula (I) is (R)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide, (S)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide, (S)—N-((4-(((1,4-dioxan-2-yl)methyl)amino)-3-fluoro-5-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide, or a pharmaceutically acceptable salt thereof.

In some embodiments, the compound of formula (I) is (S)—N-((4-(((1,4-dioxan-2-yl)methyl)amino)-3-fluoro-5-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide or a pharmaceutically acceptable salt thereof. In some embodiments, the compound of formula (I) is (R)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide or a pharmaceutically acceptable salt thereof. In some embodiments, the compound of formula (I) is (S)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide or a pharmaceutically acceptable salt thereof.

In some embodiments, the CDK inhibitor is a CDK9 inhibitor, for example, compound 2 (also known as alvocidib)

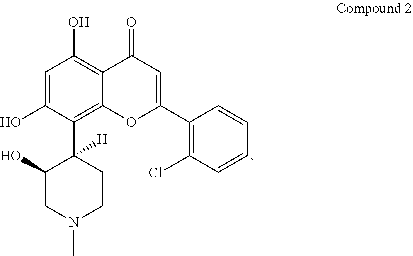 [SMILES: CN1CC[C@H](c2c(O)cc(O)c3c(=O)cc(-c4ccccc4Cl)oc23)[C@H](O)C1]

or compound 3 (also known as dinaciclib)

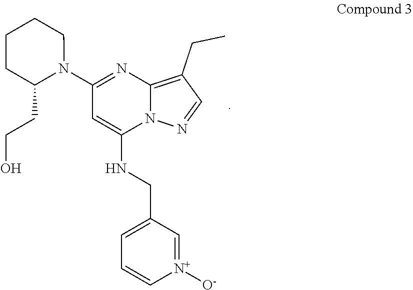 [SMILES: CCc1cnn2c(NCc3ccc[n+]([O-])c3)cc(N3CCCC[C@H]3CCO)nc12]

In some embodiments, the CDK inhibitor is the compound 2 or a pharmaceutically acceptable salt thereof.

In some embodiments, the CDK inhibitor is the compound 3 or a pharmaceutically acceptable salt thereof.

In some embodiments, the compound of formula (I) is (S)—N-((4-(((1,4-dioxan-2-yl)methyl)amino)-3-fluoro-5-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide or a pharmaceutically acceptable salt thereof, and the CDK inhibitor is the compound 2 or a pharmaceutically acceptable salt thereof.

In some embodiments, the compound of formula (I) is (S)—N-((4-(((1,4-dioxan-2-yl)methyl)amino)-3-fluoro-5-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide or a pharmaceutically acceptable salt thereof, and the CDK inhibitor is the compound 3 or a pharmaceutically acceptable salt thereof.

In some embodiments, the compound of formula (I) is (R)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide or a pharmaceutically acceptable salt thereof, and the CDK inhibitor is the compound 2 or a pharmaceutically acceptable salt thereof.

In some embodiments, the compound of formula (I) is (R)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide or a pharmaceutically acceptable salt thereof, and the CDK inhibitor is the compound 3 or a pharmaceutically acceptable salt thereof.

In some embodiments, the compound of formula (I) is (S)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide or a pharmaceutically acceptable salt thereof, and the CDK inhibitor is the compound 2 or a pharmaceutically acceptable salt thereof.

In some embodiments, the compound of formula (I) is (S)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide or a pharmaceutically acceptable salt thereof, and the CDK inhibitor is the compound 3 or a pharmaceutically acceptable salt thereof.

The disease mediated by Bcl-2 and/or CDK activity can be a cancer. The cancer includes, but is not limited to, adrenal cortical cancer, advanced cancer, anal cancer, aplastic anemia, bile duct cancer, bladder cancer, bone cancer, bone metastasis, brain/CNS tumors in adults, brain/CNS tumors in children, breast cancer, breast cancer in men, cancer in children, cancer of unknown primary, Castleman disease, cervical cancer, colon/rectum cancer, endometrial cancer, esophagus cancer, Ewing family of tumors, eye cancer, gallbladder cancer, gastrointestinal carcinoid tumors, gastrointestinal stromal tumor (GIST), gestational trophoblastic disease, Hodgkin disease, Kaposi sarcoma, kidney cancer, laryngeal and hypopharyngeal cancer, leukemia (e.g., acute lymphocytic leukemia (ALL), acute myeloid leukemia (AMIL), chronic lymphocytic leukemia (CLL), chronic myeloid leukemia (CML), chronic myelomonocytic leukemia (CMML)), liver cancer, lung cancer-non-small cell, lung cancer-small cell, lung carcinoid tumor, lymphoma of the skin, malignant mesothelioma, myelodysplastic syndrome, nasal cavity and paranasal sinus cancer, nasopharyngeal cancer, neuroblastoma, neuroectodermal tumor, peritoneal cancer, human head and neck squamous cell carcinoma (HNSCC), non-Hodgkin lymphoma (e.g. diffuse large B-cell lymphoma), non-Hodgkin lymphoma in children, Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma-adult soft tissue cancer, skin cancer-basal and squamous cell, skin cancer-melanoma, small intestine cancer, stomach cancer, testicular cancer, thymus cancer, thyroid cancer, uterine sarcoma, vaginal cancer, vulvar cancer, Waldenstrom macroglobulinemia, and Wilms Tumor. The disease mediated by Bcl-2 and/or CDK activity can also be Cardiac hypertrophy, dilated cardiomyopathy, atherosclerosis, muscle atrophy or obesity.

In some embodiments, the disease mediated by Bcl-2 and/or CDK activity is non-Hodgkin lymphoma (e.g. diffuse large B-cell lymphoma) or leukemia (e.g. myelodysplastic syndrome, chronic lymphocytic leukemia or acute myeloid leukemia). In some embodiments, the disease mediated by Bcl-2 and/or CDK activity is acute myeloid leukemia. In some embodiments, the disease mediated by Bcl-2 and/or CDK activity is diffuse large B-cell lymphoma. In some embodiments, the disease mediated by Bcl-2 and/or CDK activity is myelodysplastic syndrome. In some embodiments, the disease mediated by Bcl-2 and/or CDK activity is chronic lymphocytic leukemia.

The present invention also provides a method for the prevention and/or treatment of a cancer, which comprises administering to a subject in need thereof a therapeutically effective amount of a compound of formula (I) or a pharmaceutically acceptable salt thereof, and a therapeutically effective amount of a CDK inhibitor or a pharmaceutically acceptable salt thereof. The compound of formula (I) and the CDK inhibitor may be administered simultaneously or separately in any order. The cancer can be non-Hodgkin lymphoma (e.g. diffuse large B-cell lymphoma) or leukemia (e.g. myelodysplastic syndrome or acute myeloid leukemia). The cancer includes, but is not limited to, adrenal cortical cancer, advanced cancer, anal cancer, aplastic anemia, bile duct cancer, bladder cancer, bone cancer, bone metastasis, brain/CNS tumors in adults, brain/CNS tumors in children, breast cancer, breast cancer in men, cancer in children, cancer of unknown primary, Castleman disease, cervical cancer, colon/rectum cancer, endometrial cancer, esophagus cancer, Ewing family of tumors, eye cancer, gallbladder cancer, gastrointestinal carcinoid tumors, gastrointestinal stromal tumor (GIST), gestational trophoblastic disease, Hodgkin disease, Kaposi sarcoma, kidney cancer, laryngeal and hypopharyngeal cancer, leukemia (e.g., acute lymphocytic leukemia (ALL), acute myeloid leukemia (AML), chronic lymphocytic leukemia (CLL), chronic myeloid leukemia (CML), chronic myelomonocytic leukemia (CMML)), liver cancer, lung cancer-non-small cell, lung cancer-small cell, lung carcinoid tumor, lymphoma of the skin, malignant mesothelioma, myelodysplastic syndrome, nasal cavity and paranasal sinus cancer, nasopharyngeal cancer, neuroblastoma, neuroectodermal tumor, peritoneal cancer, human head and neck squamous cell carcinoma (HNSCC), non-Hodgkin lymphoma (e.g. diffuse large B-cell lymphoma), non-Hodgkin lymphoma in children, Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma-adult soft tissue cancer, skin cancer-basal and squamous cell, skin cancer-melanoma, small intestine cancer, stomach cancer, testicular cancer, thymus cancer, thyroid cancer, uterine sarcoma, vaginal cancer, vulvar cancer, Waldenstrom macroglobulinemia, and Wilms Tumor. In some embodiments, the cancer is acute myeloid leukemia. In some embodiments, the cancer is non-Hodgkin lymphoma (e.g. diffuse large B-cell lymphoma) or leukemia (e.g. myelodysplastic syndrome, chronic lymphocytic leukemia or acute myeloid leukemia). In some embodiments, the cancer is acute myeloid leukemia. In some embodiments, the cancer is diffuse large B-cell lymphoma. In some embodiments, the cancer is myelodysplastic syndrome. In some embodiments, the cancer is chronic lymphocytic leukemia.

The pharmaceutical composition or combination according to the present invention can further comprises a pharmaceutical carrier.

In the above composition or combination, the weight ratio of the compound of formula (I) to the CDK inhibitor can be 50:1 to 1:50, e.g. 50:1, 45:1, 40:1, 35:1, 30:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45 or 1:50.

A therapeutically effective amount of the compound of formula (I) and the CDK inhibitor can be administrated to a subject in a weight ratio of 50:1 to 1:50, e.g. 50:1, 45:1, 40:1, 35:1, 30:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45 or 1:50.

In some embodiments, the compound of formula (I) and the CDK inhibitor, e.g. the compound 2, can be administrated to a subject in a weight ratio of 20:1.

In some embodiments, the compound of formula (I) and the CDK inhibitor, e.g. the compound 3, can be administrated to a subject in a weight ratio of 3:2.

Preferred dosages for the compounds of the present invention are therapeutically effective dosages, especially those which are commercially available.

A “therapeutically effective amount” of a compound or a composition refers to an amount sufficient to cure, alleviate or partially arrest the clinical manifestations of a given disease or disorder and its complications. The amount that is effective for a particular therapeutic purpose will depend on the severity of the disease or injury as well as on the weight and general state of the subject. It will be understood that determination of an appropriate dosage may be achieved, using routine experimentation, by constructing a matrix of values and testing different points in the matrix, all of which is within the ordinary skills of a trained physician or clinical scientist. It will be appreciated that the unit content of each active agent contained in an individual dose of each dose form need not in itself constitute an effective amount since the necessary effective amount can be reached by administration of a plurality of dose units.

For example, the compound of formula (I) as part of the composition or combination according to the present invention may be orally administered to a subject at a dose of 1-2000 mg, e.g. 1-1500 mg, e.g. 1, 5, 10, 15, 20, 25, 30, 35, 40, 45, 50, 55, 60, 65, 70, 75, 80, 85, 90, 95, 100, 125, 150, 175, 200. 225, 250, 275, 300, 325, 350, 375, 400, 425, 450, 475, 500, 525, 550, 575, 600, 625, 650, 675, 700, 750, 775, 800, 825, 850, 875, 900, 925, 950, 975, 1000, 1100, 1200, 1300, 1400, 1500 mg. In some embodiments, the compound of formula (I) as part of the composition or combination according to the present invention may be orally administered to a subject, e.g. a human, at a dose of 1-1500 mg, e.g. 100-1000 mg, e.g. 100-500 mg, e.g. 200-500 mg. These doses may be administered once, twice or three times daily, Q2D (once every two days), QW (once a week), BIW (twice a week) or Q2W (once every two weeks). For example, the compound of formula (I) can be orally administered once daily.

For example, the CDK inhibitor, e.g. the compound 2, as part of the composition or combination according to the present invention, may be administered to a subject at a dose of 0.1-1000 mg, e.g. 1-500 mg, e.g. 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 15, 20, 25, 30, 35, 40, 45, 50, 55, 60, 65, 70, 75, 80, 85, 90, 95, 100, 125, 150, 175, 200. 225, 250, 275, 300, 325, 350, 375, 400, 425, 450, 475 or 500 mg. These doses may be administered once, twice or three times daily, Q2D (once every two days), QW (once a week), BIW (twice a week) or Q2W (once every two weeks). For example, the compound 2 can be intraperitoneally administered once daily. For example, the compound 3 can be orally administered once a week.

The compound of formula (I) has been disclosed in WO2018027097A1, which is incorporated herein by reference in its entirety.

The compound of formula (I) and the CDK inhibitor may be administered simultaneously or separately in any order. By “simultaneously”, it is meant that the indicated agents are administered at a same time point. However, if not administered simultaneously, it is meant that they are administered to a subject in a sequence and sufficiently close in time so as to provide the desired therapeutic effect and can act in concert. For example, a CDK inhibitor can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the compound of formula (I), to a subject in need thereof. In various embodiments, a CDK inhibitor and the compound of formula (I) are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart.

The compounds of the present invention may be administered orally. Oral administration may involve swallowing, so that the compound enters the gastrointestinal tract, and/or buccal, lingual, or sublingual administration by which the compound enters the blood stream directly from the mouth. For example, the compound of formula (I) can be administered orally. For example, the CDK inhibitor, e.g., the compound 3, can be administered orally.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches. Further, the compound or salts of the invention can be administered as a spray dried dispersion. Solid formulations for oral administration may be formulated to be immediate and/or modified release. Modified release formulations include delayed-, sustained-, pulsed-, controlled-, targeted and programmed release.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the present invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques. Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. For example, the CDK inhibitor, e.g. the compound 2, can be intraperitoneally administered daily.

Formulations for parenteral administration may be formulated to be immediate and/or modified release. Modified release formulations include delayed-, sustained-, pulsed-, controlled-, targeted and programmed release. Thus the compounds of the prevent invention may be formulated as a suspension or as a solid, semi-solid, or thixotropic liquid for administration as an implanted depot providing modified release of the active compound. Examples of such formulations include drug-coated stents and semi-solids and suspensions comprising drug-loaded poly(dl-lactic-coglycolic)acid (PGLA) microspheres.

The compounds of the present invention may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated-see, for example, J Pharm Sci, 88 (10), 955-958, by Finnin and Morgan (October 1999).

Formulations for topical administration may be formulated to be immediate and/or modified release. Modified release formulations include delayed-, sustained-, pulsed-, controlled-, targeted and programmed release.

The term “carrier” refers to a diluent, adjuvant, excipient, or vehicle with which the compound is administered. Suitable pharmaceutical carriers are described in “Remington's Pharmaceutical Sciences” by E. W. Martin. Typical pharmaceutically carriers for use are exemplified by: sugars such as lactose, sucrose, mannitol and sorbitol; starches such as cornstarch, tapioca starch and potato starch; cellulose and derivatives such as sodium carboxymethyl cellulose, ethyl cellulose and methyl cellulose; calcium phosphates such as dicalcium phosphate and tricalcium phosphate; sodium sulfate; calcium sulfate; polyvinylpyrrolidone; polyvinyl alcohol; stearic acid; alkaline earth metal stearates such as magnesium stearate and calcium stearate; stearic acid; vegetable oils such as peanut oil, cottonseed oil, sesame oil, olive oil and corn oil; non-ionic, cationic and anionic surfactants; ethylene glycol polymers; betacyclodextrin; fatty alcohols; and hydrolyzed cereal solids, as well as other non-toxic compatible fillers, binders, disintegrants, buffers, preservatives, antioxidants, lubricants, flavoring agents, and the like commonly used in pharmaceutical formulations.

The term “pharmaceutical combination” is used herein to mean a product including the active ingredients (e.g. the compound C, the CDK inhibitor) according to the present invention. The active ingredients included by the pharmaceutical combination can be present in a single entity (e.g., a single dosage form, e.g., in one injection, in one tablet or in one capsule), and thus can be administered to a subject simultaneously. The active ingredients included by the pharmaceutical combination can also be present in separate entities (e.g., one active ingredient is present in an tablet, while the other active ingredient is present in a capsule), and thus can be administered to a subject independently of each other, either simultaneously or separately with no specific time limits. If the active ingredients included by the pharmaceutical combination are present in separate entities, they can be sold independently of each other and just instruction of the possibility of their combined use is provided in the package equipment, e.g., leaflet or the like, or in other information, e.g., provided to physicians and medical staff (e.g., oral communications).

The term “combination” is used herein to meant either, simultaneous administration or any manner of separate sequential administration of a therapeutically effective amount of the compound of formula (I) and a CDK inhibitor or a pharmaceutically acceptable salt thereof. Preferably, if the administration is not simultaneous, the compounds are administered in a close time proximity to each other. Furthermore, it does not matter if the compounds are administered in the same dosage form, e.g. one compound may be administered topically and the other compound may be administered orally. Suitably, both compounds are administered orally.

The term “synergistic”, as used herein, means that the effect achieved with the methods, combinations and compositions of the present invention is greater than the sum of the effects that result from individual methods and compositions comprising the active ingredients of this invention separately. The “synergistic” effect of a combination is determined herein by the methods described in Clarke R. Issues in experimental design and endpoint analysis in the study of experimental cytotoxic agents in vivo in breast cancer and other models[J]. Breast Cancer Research & Treatment, 1997, 46(2-3):255-278, which is incorporated herein by reference in its entirety. See also Gould S E et al. Translational value of mouse models in oncology drug development. Nature medicine. 2015 21, 431-439, which is incorporated herein by reference in its entirety. See also Chou T C. Drug Combination Studies and Their Synergy Quantification Using the Chou-Talalay Method[J]. Cancer Research, 2010, 70(2): 440-446, which is incorporated herein by reference in its entirety.

The term “pharmaceutically acceptable salt” refers to a non-toxic salt commonly used in the pharmaceutical industry which may be prepared according to methods well-known in the art.

The term “pharmaceutically acceptable”, as used herein, refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

The term “prevention” refers to prophylactic administration to healthy patients to prevent the development of the conditions mentioned herein. Moreover, the term “prevention” means prophylactic administration to patients being in a pre-stage of the conditions to be treated.

The term “treatment” is understood the management and care of a patient for the purpose of combating the disease, condition or disorder.

The term “subject” refers to any animal, including mammals, such as mice, rats, other rodents, rabbits, dogs, cats, pigs, cattle, sheep, horses, primates, or humans. The preferred subjects are humans.

The term “container” means any receptacle and closure therefor suitable for storing, shipping, dispensing, and/or handling a pharmaceutical product.

The term “CDK” is an abbreviation of “cyclic-dependent kinase”, which refers to a family of proteins capable of complexing with a cyclin and capable of catalyzing phosphorylation of a substrate. Cyclin-dependent kinases (also called CDKs) are known in the art and include, for example, CDK1, CDK2, CDK3, CDK4, CDK5, CDK6, CDK7, CDK8, and CDK9.

The term “CDK inhibitor” refers to any compound that reduces, or inhibits, either partially or in full, the activity of a CDK. A CDK inhibitor may directly or indirectly reduce or inhibit the activity of one or more specific CDK(s). For example, an inhibitor of CDK4 and CDK6 can simultaneously inhibit the activity of CDK4 and CDK6.

The term “disease mediated by Bcl-2” refers to a disease in which activity of Bcl-2 leads to abnormal activity of the regulatory pathways including overexpression, mutation or relative lack of activity of other regulatory pathways in the cell that result in excessive cell proliferation, e.g. cancer.

The term “disease mediated by CDK” refers to a disease in which activity of CDK leads to abnormal activity of the regulatory pathways including overexpression, mutation or relative lack of activity of other regulatory pathways in the cell that result in excessive cell proliferation, e.g. cancer.

The use of the terms “a”, “an”, “the”, and similar referents in the context of describing the invention (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the invention and is not a limitation on the scope of the invention unless otherwise claimed. No language in the specification should be construed as indicating any non-claimed element as essential to the practice of the invention.

The term “halo” or “halogen” as used by itself or as part of another group refers to —Cl, —F, —Br, or —I.

The term “alkyl” as used by itself or as part of another group refers to unsubstituted straight- or branched-chain aliphatic hydrocarbons containing one to twelve carbon atoms, i.e., C₁₋₁₂ alkyl, or the number of carbon atoms designated, e.g., a C₁ alkyl such as methyl, a C₂ alkyl such as ethyl, a C₃ alkyl such as propyl or isopropyl, a C₁₋₃ alkyl such as methyl, ethyl, propyl, or isopropyl, and so on. In one embodiment, the alkyl group is a straight chain C₁₋₆ alkyl group. In another embodiment, the alkyl group is a branched chain C₃₋₆ alkyl group. In another embodiment, the alkyl group is a straight chain C₁₋₄ alkyl group. In another embodiment, the alkyl group is a branched chain C₃₋₄ alkyl group. In another embodiment, the alkyl group is a straight or branched chain C₃₋₄ alkyl group. In another embodiment, the alkyl group is partially or completely deuterated, i.e., one or more hydrogen atoms of the alkyl group are replaced with deuterium atoms. Non-limiting exemplary C₁₋₁₂ alkyl groups include methyl, —CD₃, ethyl, propyl, isopropyl, butyl, sec-butyl, tert-butyl, iso-butyl, 3-pentyl, hexyl, heptyl, octyl, nonyl, and decyl. Non-limiting exemplary C₁₋₄ alkyl groups include methyl, ethyl, propyl, isopropyl, butyl, sec-butyl, tert-butyl, and iso-butyl.

The term “optionally substituted alkyl” as used by itself or as part of another group refers to an alkyl that is unsubstituted or substituted with one, two, or three substituents independently selected from the group consisting of halo, nitro, cyano, hydroxy, alkoxy, amino, alkylamino, dialkylamino, and optionally substituted aryl. In one embodiment, the optionally substituted alkyl is substituted with two substituents. In another embodiment, the optionally substituted alkyl is substituted with one substituent. In another embodiment, the optionally substituted alkyl is unsubstituted. Non-limiting exemplary optionally substituted alkyl groups include —CH₂Ph, —CH₂CH₂NO₂, —CH₂CH₂OH, —CH₂CH₂OCH₃, and —CH₂CH₂F.

The term “cycloalkyl” as used by itself or as part of another group refers to unsubstituted saturated or partially unsaturated, e.g., containing one or two double bonds, cyclic aliphatic hydrocarbons containing one to three rings having from three to twelve carbon atoms, i.e., C₃₋₁₂ cycloalkyl, or the number of carbons designated. In one embodiment, the cycloalkyl group has two rings. In one embodiment, the cycloalkyl group has one ring. In another embodiment, the cycloalkyl group is a C₃₋₈ cycloalkyl. In another embodiment, the cycloalkyl group is a C₃₋₆ cycloalkyl. In another embodiment, the cycloalkyl group is a C₃₋₅ cycloalkyl. The term “cycloalkyl” is meant to include groups wherein a ring —CH₂— is replaced with a —C(═O)—. Non-limiting exemplary cycloalkyl groups include cyclopropyl, cyclobutyl, cyclopentyl, cyclohexyl, cycloheptyl, cyclooctyl, norbornyl, decalin, adamantyl, cyclohexenyl, cyclopentenyl, cyclopentanone, spiro[3.3]heptane, and bicyclo[3.3.1]nonane.

The term “optionally substituted cycloalkyl” as used by itself or as part of another group refers to a cycloalkyl that is either unsubstituted or substituted with one, two, or three substituents independently selected from the group consisting of halo, nitro, cyano, hydroxy, alkyl, alkoxy, amino, alkylamino, dialkylamino, haloalkyl, and heterocyclo. In one embodiment, the optionally substituted cycloalkyl is substituted with two substituents. In another embodiment, the optionally substituted cycloalkyl is substituted with one substituent. In another embodiment, the optionally substituted cycloalkyl is unsubstituted.

The term “haloalkyl” as used by itself or as part of another group refers to an alkyl substituted by one or more fluorine, chlorine, bromine and/or iodine atoms. In one embodiment, the alkyl group is substituted by one, two, or three fluorine and/or chlorine atoms. In another embodiment, the haloalkyl group is a C₁₋₄ haloalkyl group. Non-limiting exemplary haloalkyl groups include fluoromethyl, 2-fluoroethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The term “alkoxy” as used by itself or as part of another group refers to an optionally substituted alkyl attached to a terminal oxygen atom. In one embodiment, the alkoxy group is a C₁₋₆ alkyl attached to a terminal oxygen atom. In another embodiment, the alkoxy group is a C₁₋₄ alkyl attached to a terminal oxygen atom. Non-limiting exemplary alkoxy groups include methoxy, ethoxy, and tert-butoxy.

The term “aryl” as used by itself or as part of another group refers to unsubstituted monocyclic or bicyclic aromatic ring systems having from six to fourteen carbon atoms, i.e., a C₆₋₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is phenyl or naphthyl.

The term “optionally substituted aryl” as used herein by itself or as part of another group refers to an aryl that is either unsubstituted or substituted with one to five substituents independently selected from the group consisting of halo, nitro, cyano, hydroxy, alkyl, alkoxy, amino, alkylamino, dialkylamino, haloalkyl, and heterocyclo. In one embodiment, the optionally substituted aryl is an optionally substituted phenyl. In another embodiment, the optionally substituted phenyl has one substituent. In another embodiment, the optionally substituted phenyl is unsubstituted. Non-limiting exemplary substituted aryl groups include 2-methylphenyl, 2-methoxyphenyl, 2-fluorophenyl, and 4-chlorophenyl.

The term “heterocyclo” as used by itself or as part of another group refers to unsubstituted saturated and partially unsaturated, e.g., containing one or two double bonds, cyclic groups containing one, two, or three rings having from three to fourteen ring members, i.e., a 3- to 14-membered heterocyclo, wherein at least one carbon atom of one of the rings is replaced with a heteroatom. The term “heterocyclo” is meant to include cyclic ureido groups such as imidazolidinyl-2-one, cyclic amide groups such as β-lactam, γ-lactam, δ-lactam and ε-lactam, and cyclic carbamate groups such as oxazolidinyl-2-one. In one embodiment, the heterocyclo group is a 4-, 5-, 6-, 7- or 8-membered cyclic group containing one ring and one or two oxygen and/or nitrogen atoms. In one embodiment, the heterocyclo group is a 5- or 6-membered cyclic group containing one ring and one or two nitrogen atoms. In one embodiment, the heterocyclo group is an 8-, 9-, 10-, 11-, or 12-membered cyclic group containing two rings and one or two nitrogen atoms. In one embodiment, the heterocyclo group is a 4- or 5-membered cyclic group containing one ring and one oxygen atom. The heterocyclo can be optionally linked to the rest of the molecule through a carbon or nitrogen atom. Non-limiting exemplary heterocyclo groups include 1,4-dioxane, 2-oxopyrrolidin-3-yl, 2-imidazolidinone, piperidinyl, morpholinyl, piperazinyl, pyrrolidinyl, 8-azabicyclo[3.2.1]octane (nortropane), 6-azaspiro[2.5]octane, 6-azaspiro[3.4]octane, indolinyl, indolinyl-2-one, and 1,3-dihydro-2H-benzo[d]imidazol-2-one.

The term “optionally substituted heterocyclo” as used herein by itself or part of another group refers to a heterocyclo that is either unsubstituted or substituted with one, two, or three substituents independently selected from the group consisting of halo, nitro, cyano, hydroxy, alkyl, alkoxy, amino, alkylamino, dialkylamino, haloalkyl, and heterocyclo. Non-limiting exemplary optionally substituted heterocyclo groups include

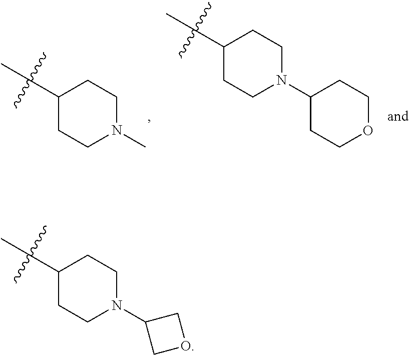

The term “alkylamino” as used by itself or as part of another group refers to —NHR¹⁰ wherein R¹⁰ is C₁₋₆ alkyl. In one embodiment, R¹⁰ is C₁₋₄ alkyl. Non-limiting exemplary alkylamino groups include —N(H)CH₃ and —N(H)CH₂CH₃.

The term “dialkylamino” as used by itself or as part of another group refers to —NR^{11a}R^{11b}, wherein R^{11a} and R^{11b} are each independently C₁₋₆ alkyl. In one embodiment, R^{11a} and R^{11b} are each independently C₁₋₄ alkyl. Non-limiting exemplary dialkylamino groups include —N(CH₃)₂ and —N(CH₃)CH₂CH(CH₃)₂.

The term “(cycloalkyl)alkyl” as used by itself or as part of another group refers to an alkyl substituted with one optionally substituted cycloalkyl group. In one embodiment, the (cycloalkyl)alkyl is a C₁₋₄ alkyl substituted with one optionally substituted C₃₋₆ cycloalkyl. In one embodiment, the optionally substituted cycloalkyl group is substituted with a heterocyclo group. Non-limiting exemplary (cycloalkyl)alkyl groups include

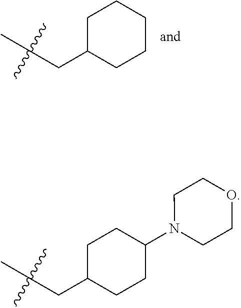 [SMILES: *CC1CCCCC1 ; *CC1CCC(N2CCOCC2)CC1]

The term “(heterocyclo)alkyl” as used by itself or as part of another group refers to an alkyl substituted with one optionally substituted heterocyclo group. In one embodiment, the (heterocyclo)alkyl is a C₁₋₄ alkyl substituted with one optionally substituted 4- to 6-membered heterocyclo group. The heterocyclo can be linked to the alkyl group through a carbon or nitrogen atom. Non-limiting exemplary (heterocyclo)alkyl groups include

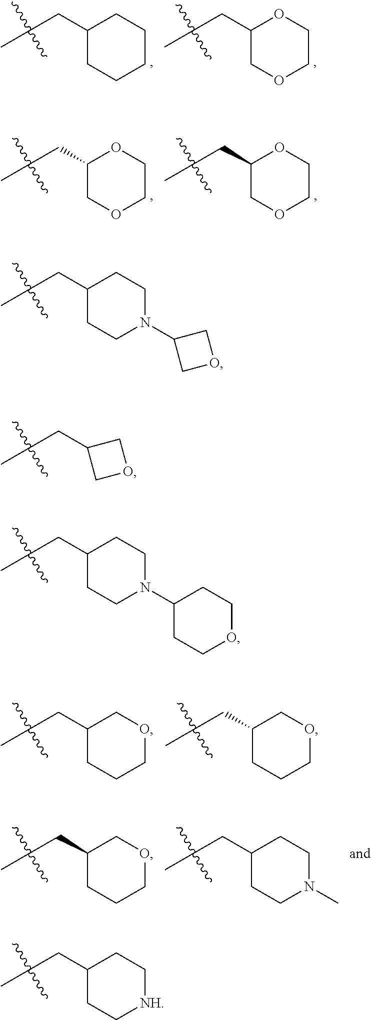

The term “heteroalkyl” as used by itself or part of another group refers to unsubstituted straight- or branched-chain aliphatic hydrocarbons containing from six to twelve chain atoms, i.e., 6- to 12-membered heteroalkyl, or the number of chain atoms designated, wherein at least two —CH₂— groups are independently replaced with —O—, —N(H)—, or —S—. The —O—, —N(H)—, or —S— can independently be placed at any interior position of the aliphatic hydrocarbon chain so long as each —O—, N(H)—, or —S— group is separated by at least two —CH₂— groups. In one embodiment, two —CH₂— groups are replaced with two —O— groups. In another embodiment, three —CH₂— groups are replaced with three —O— groups. Non-limiting exemplary heteroalkyl groups include —CH₂CH₂OCH₂CH₂OCH₃, —CH₂CH₂OCH₂CH₂N(H)CH₃, and —CH₂CH₂OCH₂CH₂OCH₂CH₂OCH₃.

The pharmaceutical compositions and preparations are manufactured by conventional mixing, granulating, dragee-making, dissolving, or lyophilizing processes.

The compounds of the present invention can be present as pharmaceutical acceptable salts. If these compounds have, for example, at least one basic center, they can form acid addition salts. Corresponding acid addition salts can also be formed having, if desired, an additionally present basic center. The compounds having at least one acid group (for example COOH) can also form salts with bases. Corresponding internal salts may furthermore be formed, if a compound comprises e. g. both a carboxy and an amino group.

The compounds of the present invention can be present in form of a hydrate or include other solvents used for crystallization.

The compounds of the present invention can be present in form of one or more polymorphic forms.

The present invention further includes all possible stereoisomers and geometric isomers of the compounds of the present invention. The present invention includes both racemic compounds and optically active isomers. When a compound is desired as a single enantiomer, it can be obtained either by resolution of the final product or by stereospecific synthesis from either isomerically pure starting material or use of a chiral auxiliary reagent, for example, see Z. Ma et al., Tetrahedron: Asymmetry, 8(6), pages 883-888 (1997). Resolution of the final product, an intermediate, or a starting material can be achieved by any suitable method known in the art. Additionally, in situations where tautomers of the compounds of structural formula (I) are possible, the present invention is intended to include all tautomeric forms of the compounds.

The present invention includes all pharmaceutically acceptable isotopically-labeled compounds, e.g. compound of formula (I), wherein one or more atoms are replaced by atoms having the same atomic number, but an atomic mass or mass number different from the atomic mass or mass number usually found in nature.

Examples of isotopes suitable for inclusion in the compounds of the present invention comprises isotopes of hydrogen, such as ²H and ³H, carbon, such as ¹¹C, ¹³C and ¹⁴C, nitrogen, such as ¹³N and ¹⁵N, oxygen, such as ¹⁵O, ¹⁷O and ¹⁸O.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

The compounds of the present invention can be present as prodrugs. Thus, certain derivatives which may have little or no pharmacological activity themselves can, when administered into or onto the body, be converted into compounds of the present invention having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs can, for example, be produced by replacing appropriate functionalities present in the compounds of the present invention with certain moieties known to those skilled in the art as ‘pro-moieties’ as described, for example, in “Design of Prodrugs” by H Bundgaard (Elsevier, 1985).

Some examples of such prodrugs include:

(i) where the compound contains an alcohol functionality (—OH), an ether thereof, for example, replacement of the hydrogen with (C₁-C₆)alkanoyloxymethyl;

(ii) where the compound contains a secondary amino functionality, an amide thereof, for example, replacement of hydrogen with (C₁-C₁₀)alkanoy.

The abbreviations “p.o” (i.e. orally), “i.p.” (i.e. intraperitoneally), “q.d” or “QD” (i.e. daily), “qw” (i.e. once every week) and the like are used to describe the route of administration or the dosage regiment in their general meanings.

The person skilled in the pertinent art is fully enabled to select a relevant test model to prove the efficacy of the composition of the present invention in the hereinbefore and hereinafter indicated therapeutic indications. Representative studies are carried out with a combination of the compound 1 and alvocidib.

All publications and patent applications mentioned herein are herein incorporated by reference in their entireties to the same extent as if each individual publication or patent application is specifically and individually indicated to be incorporated by reference.

It has surprisingly been found that, the compound 1 and alvocidib achieves greater therapeutic effect than the administration of the compound 1 or alvocidib and significantly reduces the tumor growth and increase the response rate in a OCI-AML-3 AML, SKM-1 MDS and U2932 DLBCL xenograft model, which exhibits significant synergistic effect. In U2932 DLBCL xenograft model, the compound 1 enhanced tumor inhibition of alvocidib significantly with the T/C value (average tumor volumes in treatment group vs the vehicle control group) improved from 30.9% to 5.8%. Interestingly, the combination treatment showed a 100% response rate with 20% of complete tumor regression (CR) and 80% partial regression (PR). In contrast, no CR or PR occurred in alvocidib single arm. Similar synergistic antitumor activity was observed in the myelodysplastic syndromes SKM-1 and AML OCI-AML-3 xenograft models with T/C values 2.9% and 3.4% recorded. Strikingly, in the OCI-AML-3 xenograft experiment, the combined treatment arm showed a 60% response rate with 40% CR and 20% PR. No CR or PR occurred in single arms.

Further benefits can be that lower doses of the individual drugs to be combined according to the present invention can be used to reduce the dosage, for example, that the dosages need not only often be smaller but are also applied less frequently, or can be used to diminish the incidence of side effects.

### BRIEF DESCRIPTION OF THE DRAWINGS

FIG. 1 shows antitumor activity of Compound 1 as a single agent or in combination with alvocidib in subcutaneous human OCI-AML-3 AML xenograft model in embodiment 2.

FIG. 2 shows body weight change (%) of mice under the treatment of Compound 1 and alvocidib in subcutaneous human OCI-AML-3 AML xenograft model in embodiment 2.

FIG. 3 shows antitumor activity of Compound 1 as a single agent or in combination with alvocidib in subcutaneous human SKM-1 MDS xenograft model in embodiment 3.

FIG. 4 shows body weight change (%) of mice under the treatment of Compound 1 and alvocidib in subcutaneous human SKM-1 MDS xenograft model in embodiment 3.

FIG. 5 shows antitumor activity of Compound 1 as a single agent or in combination with alvocidib in subcutaneous human U2932 DLBCL xenograft model in embodiment 4.

FIG. 6 shows body weight change (%) of mice under the treatment of Compound 1 and alvocidib in subcutaneous human U2932 DLBCL xenograft model in embodiment 4.

FIG. 7 shows the antiproliferative effect of Compound 1 as a single agent or in combination with alvocidib in U2932 cell line in embodiment 5.

FIG. 8 shows the antiproliferative effect of Compound 1 as a single agent or in combination with alvocidib in OCI-AML3 cell line in embodiment 5.

FIG. 9 shows the antitumor activity of Compound 1 as a single agent or in combination with dinaciclib in subcutaneous U2932 human DLBCL xenograft in embodiment 6.

### DETAILED DESCRIPTION OF THE PREFERRED EMBODIMENTS

The following examples further illustrate the present invention, but the present invention is not limited thereto.

### Embodiment 1: Synthesis of Compound 1

Compound 1: (S)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide, was synthesized according to WO2018027097A1.

### Embodiment 2

Cell Culture and Animal Studies

OCI-AML-3 human acute myeloid leukemia and U2932 human diffuse large B-cell lymphoma cell lines were maintained in vitro as suspension in RPMI 1640 medium supplemented with 10% fetal bovine serum, 100 U/mL penicillin and 100 g/mL streptomycin. SKM-1 human Myelodysplastic Syndromes (MDS) were maintained in vitro as suspension cultures in RPMI 1640 medium supplemented with 20% fetal bovine serum, 100 U/mL penicillin and 100 g/mL streptomycin. Cells were maintained at 37° C. in an atmosphere of 5% CO₂ in air and passaged twice weekly. Tumor cells growing in an exponential growth phase were harvested and counted for tumor inoculation. Each mouse was inoculated subcutaneously at the right flank region with OCI-AML-3 tumor cells (5×10⁶) in 0.2 mL of PBS with 50% matrigel, SKM-1 tumor cells (3×10⁷) in 0.2 mL of PBS with 50% matrigel, U2932 tumor cells (1×10⁷) in 0.2 mL of PBS with 50% matrigel, for tumor development.

Observations and Data Collection

After tumor cell inoculation, the animals were checked daily for morbidity and mortality. At the time of routine monitoring, the animals were checked for any effects of tumor growth and treatments on normal behavior such as mobility, visual estimation of food and water consumption, body weight gain/loss (body weights were measured twice weekly), eye/hair matting and any other abnormal effect. Death and observed clinical signs were recorded on the basis of the numbers of animals within each subset. The entire procedures of dosing as well as tumor and body weight measurement were conducted in a Laminar Flow Cabinet.

Tumor volumes were measured twice weekly in two dimensions using a caliper, and the volume was expressed in mm³ using the formula:

Tumor Volume (mm³)=0.5a×b²

where a and b are the long and short diameters of the tumor, respectively.

Relative tumor volume (RTV) was calculated using the following formula:

RTV=Vₜ/V₁

where V₁ and Vₜ are the average tumor volume on the first day of treatment (day 1) and the average tumor volume on a certain time point (day t).

Synergy score was calculated using the following formula described in Clarke R. Issues in experimental design and endpoint analysis in the study of experimental cytotoxic agents in vivo in breast cancer and other models[J]. Breast Cancer Research & Treatment, 1997, 46(2-3):255-278, which is incorporated by reference in its entirety:

Synergy score=((A/C)×(B/C))/(AB/C);

where A: response to treatment A; B: response to treatment B; C: response to vehicle control; AB: combination of treatment A and B. Synergy score>1, synergistic; score=1, additive; score<1, antagonistic.

Blood and tumor samples were collected 4 hours after the final dose.

Standard NCI procedures were used to calculate tumor parameters. Percent tumor growth inhibition (% T/C) was calculated as the mean RTV of treated tumors (T) divided by the mean RTV of control tumors (C)×100%. The percentage T/C value is an indication of antitumor effectiveness: a value of T/C<42% is considered significant antitumor activity by the NCI. A T/C value<10% is considered to indicate highly significant antitumor activity, and is the level used by the NCI to justify a clinical trial if toxicity and certain other requirements are met (termed DN-2 level activity). A body weight loss (mean of group) of greater than 20%, or greater than 20% of drug deaths are considered to indicate an excessively toxic dosage.

Result

Synergistic Antitumor Effect in Human OCI-AML-3 AML Xenograft Model

Alvocidib is a flavonoid alkaloid CDK9 kinase inhibitor, which is the first CDKi under clinical development for the treatment of acute myeloid leukemia. In this study, efficacy of combination treatment with Compound 1 and alvocidib was evaluated in human OCI-AML-3 AML xenograft model (Study No. SZ-EF-42-2018). Dose treatments were started when the mean tumor volume reached 95 mm³, the dose treatment start day was defined as day 1.

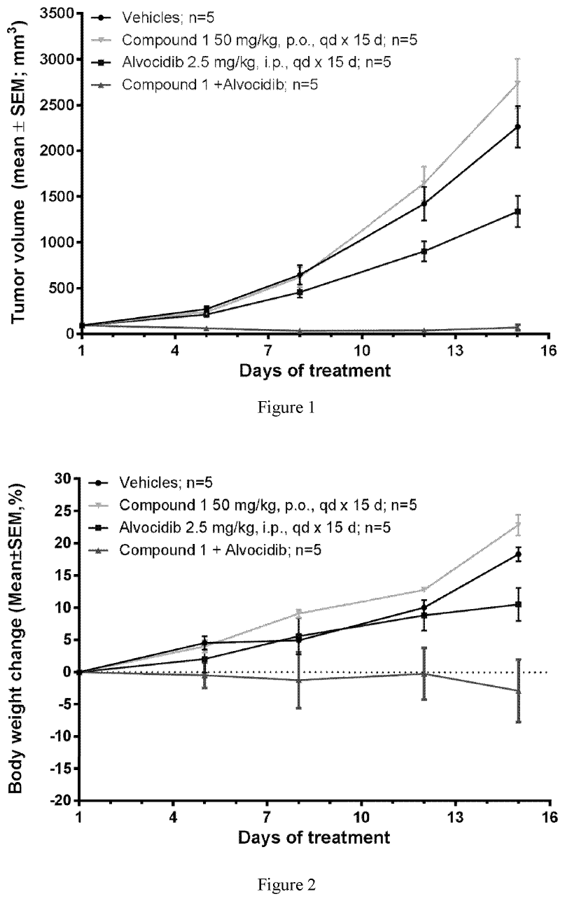

As shown in FIG. 1 and Table 1, treatment with Compound 1 as a single agent at a dose of 50 mg/kg, q.d×15 d, p.o. showed no antitumor activity whereas alvocidib at 2.5 mg/kg, q.d×15 d, i.p. demonstrated moderate antitumor activity in OCI-AML-3 xenograft model (T/C: 58.8%, p<0.05 vs vehicle). Combination treatment with Compound 1 (50 mg/kg, q.d×15 d, p.o.) and alvocidib (2.5 mg/kg, q.d×15 d, i.p) exhibited synergistic antitumor activity, achieving a T/C value of 3.4% (p<0.001 vs vehicle; p<0.001 vs Compound 1; p<0.001 vs alvocidib) on d15. The synergy score was 22.21 indicating synergistic effects. Animals from the combination group achieved 1/5 PR (partial regression) and 2/5 CR (complete regression) with an overall response rate of 60% (Table 1). No obvious body weight loss was observed under all treatment arms as shown in FIG. 2.

TABLE 1
Efficacy of Compound 1 in combination with alvocidib in
subcutaneous human OCI-AML-3 AML xenograft
model in SCID mice (SZ-EF-42-2018)

| | | | Synergy | |
|---|---|---|---|---|
| | | T/C(%)@ | score @ | Tumor status @ |
| Treatment | RTV@ D15 | D15 | D15 | D15 |
| Vehicle | 23.8 ± 2.3 | — | — | — |
| Compound 1 | 28.6 ± 2.3 | 120.2 | — | — |
| 50 mg/kg | | | | |
| Alvocidib | 14.0 ± 1.5** | 58.8 | — | — |
| 2.5 mg/kg | | | | |
| Compound | 0.8 ± 0.3***^{###&&&} | 3.4 | 22.21 | 2/5 CR, 1/5 PR |
| 1 + Alvocidib | | | | (ORR = 100%) |

**p < 0.01 vs vehicle control group;
***p < 0.001 vs vehicle control group;
^{###}p < 0.001 vs Compound 1 group;
^{&&&}p < 0.001 vs Alvocidib group;
Synergy score > l, synergistic;
score = 1, additive;
score < 1, antagonistic;
CR: complete regression;
PR: partial regression.
ORR: overall response rate

### Embodiment 3

Synergistic Antitumor Effect in Human SKM-1 MDS Xenograft Model

In this study, efficacy of combination treatment with Compound 1 and alvocidib was evaluated in human SKM-1 MDS xenograft model (Study No. SZ-EF-59-2018). Dose treatments were started when the mean tumor volume reached 125 mm³, the dose treatment start day was defined as day 1.

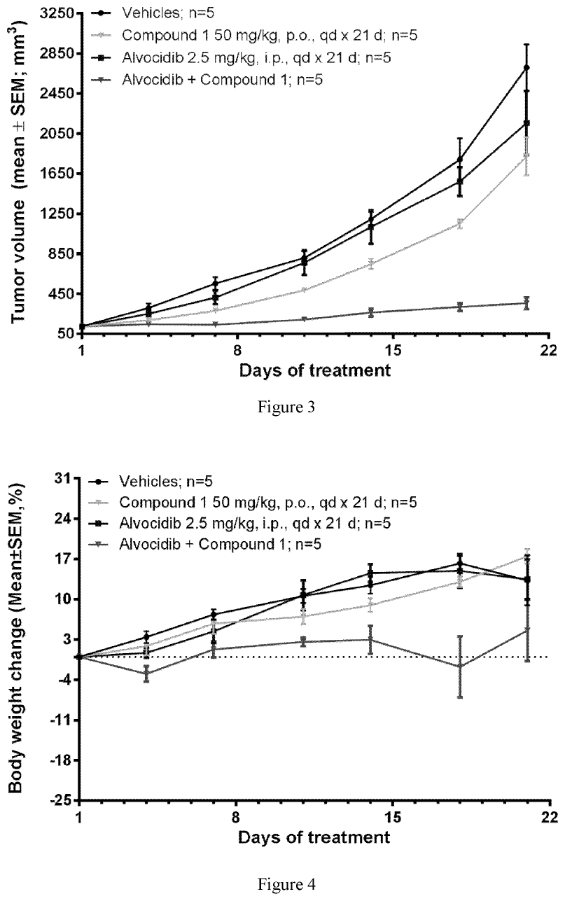

As shown in FIG. 3 and Table 2, treatment with Compound 1 as a single agent at a dose of 50 mg/kg, q.d×21 d, p.o. showed moderate antitumor activity in SKM-1 xenograft model, resulting in T/C values of 68.3% (p<0.05 vs vehicle) on d21. Alvocidib at 2.5 mg/kg, q.d×21 d, i.p. demonstrated no antitumor activity in SKM-1 xenograft model, resulting in T/C values of 78.5% (p>0.05 vs vehicle) on d21. Combination treatment with Compound 1 (50 mg/kg, q.d×21 d, p.o.) and alvocidib (2.5 mg/kg, q.d×21 d, i.p.) exhibited synergistic antitumor activity, achieving a T/C value of 13.3% (p<0.001 vs vehicle; p<0.001 vs Compound 1; p<0.001 vs alvocidib) on d21. The synergy score was 3.99 indicating synergistic effects. No obvious body weight loss was observed under all treatments (FIG. 4).

TABLE 2
Efficacy of Compound 1 in combination with alvocidib in
subcutaneous human SKM-1 MDS xenograft model
in SCID mice (SZ-EF-59-2018)

| | | | Synergy |
|---|---|---|---|
| Treatment | RTV@ D21 | T/C(%)@D21 | score @ D21 |
| Vehicles | 21.9 ± 1.5 | — | — |
| Compound 1 | 14.7 ± 1.5* | 67.3 | — |
| Alvocidib | 17.2 ± 2.1 | 78.5 | — |
| Alvocidib + Compound 1 | 2.9 ± 0.5***^{##$$$} | 13.3 | 3.99 |

*p < 0.05;
***p < 0.001 vs vehicle control group;
^{###}p < 0.001 vs Compound 1 group;
^{$$$}p < 0.001 vs Alvocidib group;
Synergy score > l, synergistic;
score = 1, additive;
score < 1, antagonistic;
CR: complete regression;
PR: partial regression.

### Embodiment 4

Synergistic Antitumor Effect in Human U2932 DLBCL Xenograft Model

In this study, efficacy of combination treatment with Compound 1 and alvocidib was evaluated in human U2932 DLBCL xenograft model (Study No. SZ-EF-62-2018). Dose treatments were started when the mean tumor volume reached 125 mm³, the dose treatment start day was defined as day 1.

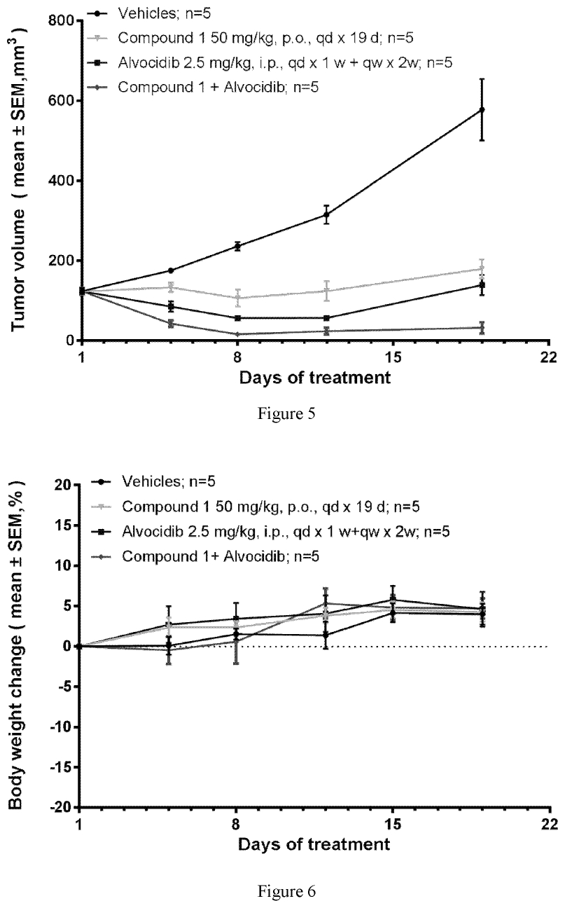

As shown in FIG. 5 and Table 3, treatment with Compound 1 as a single agent at a dose of 50 mg/kg, q.d×19 d, p.o. showed antitumor activity in SKM-1 xenograft model, resulting in T/C values of 30.9% (p<0.05 vs vehicle) on d19. Alvocidib at 2.5 mg/kg, q.d×7 d and q.w×2 w, i.p. demonstrated antitumor activity in SKM-1 xenograft model, resulting in T/C values of 25.2% (p<0.05 vs vehicle) on d19. Combination treatment with Compound 1 (50 mg/kg, q.d×19 d, p.o.) and alvocidib (2.5 mg/kg, q.d×7 d and q.w×2 w) exhibited synergistic antitumor activity, achieving a T/C value of 5.8% (p<0.05 vs vehicle; p<0.01 vs Compound 1; p<0.05 vs alvocidib) on d19. The synergy score was 1.34 indicating synergistic effects. Animals from the combination group achieved 4/5 PR (partial regression) and 1/5 CR (complete regression) with an overall response rate of 100% (Table 3). No obvious body weight loss was observed under all treatments (FIG. 6).

TABLE 3
Efficacy of Compound 1 in combination with alvocidib in
subcutaneous human U2932 DLBCL xenograft model
in SCID mice (SZ-EF-62-2018)

| | | | Synergy | |
|---|---|---|---|---|
| | | | score | Tumor |
| Treatment | RTV@ D19 | T/C(%)@D19 | @ D19 | status @ D19 |
| Vehicles | 4.71 ± 0.64 | — | — | — |
| Compound 1 | 1.46 ± 0.17* | 30.9 | — | — |
| Alvocidib | 1.19 ± 0.27* | 25.2 | — | — |
| Alvocidib + | 0.27 ± 0.11*^{##@} | 5.8 | 1.34 | 1/5 CR, 4/5 PR |
| Compound 1 | | | | (ORR = 100%) |

*p < 0.05 vs vehicle control group;
^{##}p < 0.01 vs Compound 1 group;
^{@}p < 0.05 vs Alvocidib group;
Synergy score > l, synergistic;
score = 1, additive;
score < 1, antagonistic;
CR: complete regression;
PR: partial regression.
ORR: overall response rate

### Embodiment 5: Antiproliferative Activity of Compound 1 Combined With CDK9 Inhibitor Alvocidib in Human DLBCL and AML Cell Lines

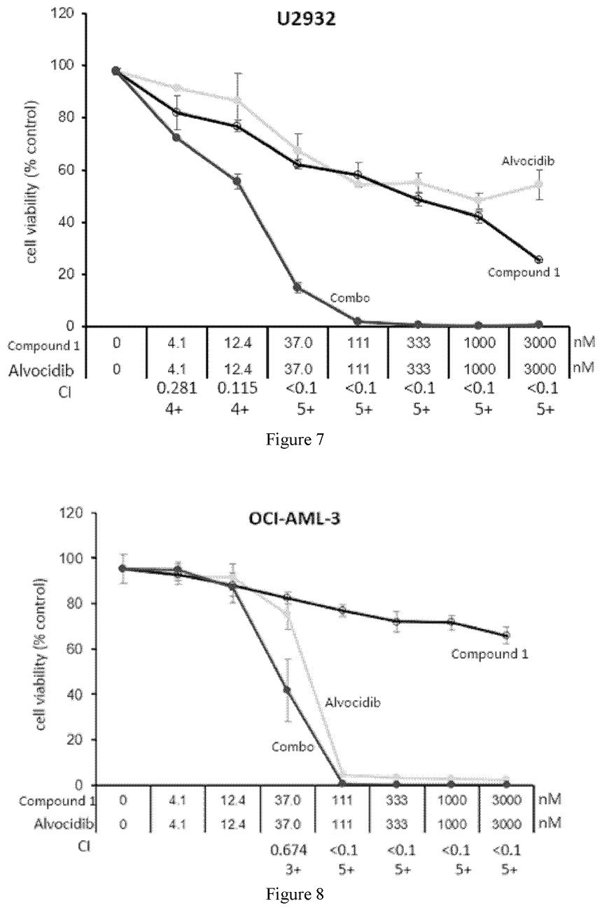

The inhibition of cell proliferation by the combination with Compound 1 and alvocidib, a CDK9 inhibitor, was evaluated in human DLBCL cell line U2932 and AML cell line OCI-AML-3 (FIGS. 7 and 8).

Cell proliferative (i.e., viability) curves were plotted using Graphpad Prism 6.0 software (Golden software, Golden, Colo., USA). In the combination treatment, combination index (CI) values were calculated using the CalcuSyn software (BIOSOFT, UK) to further analyze the combination effect of indicated drugs (Chou T C. Drug Combination Studies and Their Synergy Quantification Using the Chou-Talalay Method[J]. Cancer Research, 2010, 70(2): 440-446). CalcuSyn is a professional mixed-drugs analysis software, it accurately calculates the interaction of combined drugs, including synergy, addition, and antagonism. If the CI value of two combined drugs is <1, the results indicate that these two drugs have a synergistic effect. If the CI value is 1, the results indicate that these two drugs have an additive effect. If the CI value is >1, the results indicate that these two drugs have an antagonistic effect. In the cell proliferative curves of FIG. 7 or 8, CI<0.1 was labeled as 5+ indicating a very strong synergistic effect. CI between 0.1 and 0.3 labeled as 4+ indicating a strong synergistic effect. CI between 0.3 and 0.7 labeled as 3+ indicating a medium synergistic effect.

The results showed that the combination treatment significantly enhanced the antiproliferative activity in comparison with single agents. Synergy with CI<0.9 was achieved at low concentrations of tested agents, suggesting the synergistic effect.

### Embodiment 6: Synergistic Antitumor Activity of Compound 1 Plus Dinaciclib in Subcutaneous Human U2932 DLBCL Xenograft Models (Study No. SZ-EF-37-2019)

To confirm the synergistic antitumor effect between Compound 1 and CDK9 inhibitor in DLBCL, we applied combination therapy with Compound 1 plus CDK9 inhibitor dinaciclib in the same human U2932 DLBCL xenograft model, which was established according to embodiment 2.

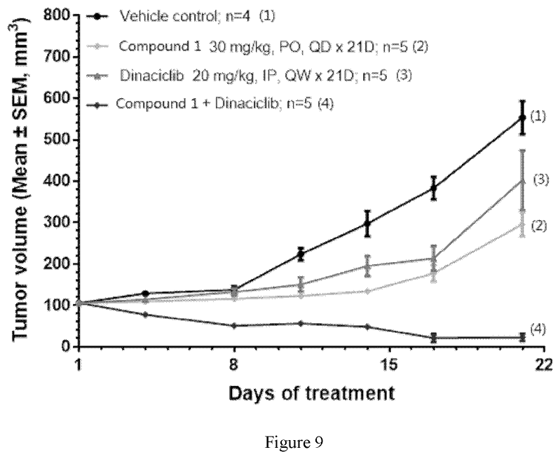

As shown in FIG. 9, Table 4, single agent treatment with dinaciclib at 20 mg/kg showed no antitumor activity with a T/C value of 71.55% (P>0.05 vs vehicle). Treatment with Compound 1 at a dose of 30 mg/kg demonstrated moderate antitumor activity, resulting in a T/C values of 53.26% (P<0.01 vs vehicle). Combination treatment with Compound 1 and dinaciclib exhibited synergistic antitumor activity, achieving a T/C value of 4.24% (P<0.01 vs vehicle; P<0.01 vs Compound 1; P<0.05 vs dinaciclib); and synergy score of 8.90. Animals achieved 1/4 CR and 3/4 PR (100% response rate) in the Compound 1 and dinaciclib combination group. In contrast, no CR or PR was recorded in the single arms.

TABLE 4
Efficacy of Compound 1 in combination with dinaciclib
in U2932 DLBCL xenograft model in
SCID mice (SZ-EF-37-2019)

| | | | Synergy | |
|---|---|---|---|---|
| | RTV @ D21 | T/C (%) | score | |
| Treatment | (Mean + SEM) | @ D21 | @ D21 | mRECIST |
| Vehicle control | 5.23 ± 0.28 | — | — | 5/5 PD |
| Compound 1 | 2.79 ± 0.22** | 53.26 | — | 5/5 SD |
| Dinaciclib | 3.74 ± 0.58 | 71.55 | — | 3/5 SD, 2/5 PD |
| Compound 1 + | | | | |
| Dinaciclib | 0.22 ± 0.08**^{##&} | 4.24 | 8.98 | 1/4 CR, 3/4 PR |

**P < 0.01 vs vehicle group;
^{##}P < 0.01 vs Compound 1 group;
^{&}P < 0.05 vs dinaciclib group.
Synergy score > l, synergistic;
score = 1, additive;
score < 1, antagonistic.
PD: progressive disease;
SD: stable disease;
CR: complete response;
PR: partial response.

It is to be understood that the foregoing description of the preferred embodiments is intended to be purely illustrative of the principles of the invention, rather than exhaustive thereof, and that changes and variations will be apparent to those skilled in the art, and that the present invention is not intended to be limited other than expressly set forth in the following claims.

1. A pharmaceutical composition comprising
(i) a compound of formula (I) or a pharmaceutically acceptable salt thereof;
(ii) a CDK inhibitor or a pharmaceutically acceptable salt thereof; 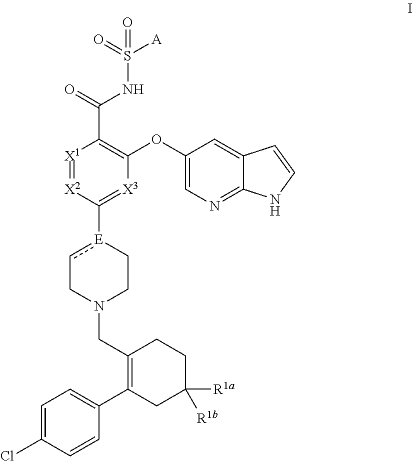
wherein, A is selected from the group consisting of 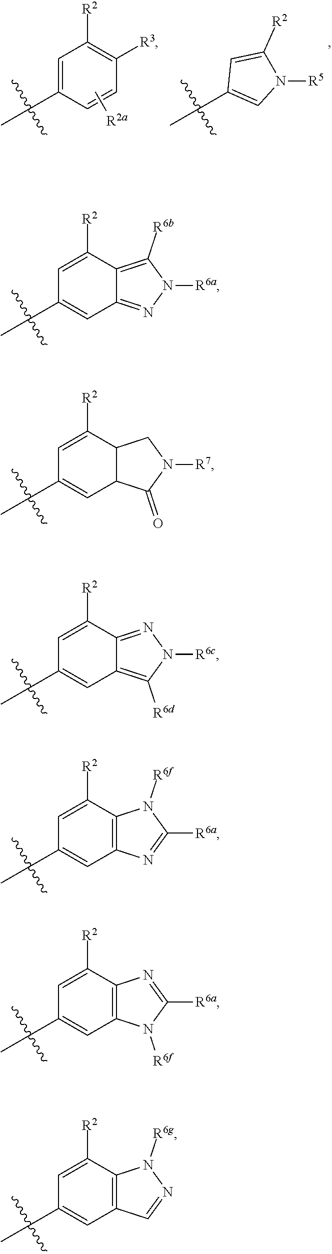 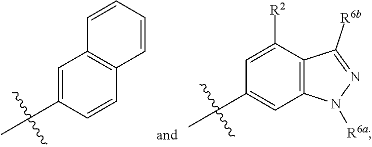
E is a carbon atom and [symbol] is a double bond; or
E is a —C(H)— and [symbol] is a single bond, or
E is a nitrogen atom and [symbol] is a single bond;
X¹, X², and X³ are each independently selected from the group consisting of —CR⁸═ and —N═;
R^{1a} and R^{1b} taken together with the carbon atom to which they are attached form a 3-, 4-, or 5-membered optionally substituted cycloalkyl; or
R^{1a} and R^{1b} taken together with the carbon atom to which they are attached form a 4- or 5-membered optionally substituted heterocyclo;
each of R² is independently selected from the group consisting of —NO₂, —SO₂CH₃, and —SO₂CF₃;
each of R^{2a} is independently selected from the group consisting of hydrogen and halogen;
R³ is selected from the group consisting of hydrogen, —CN, —C≡CH, and —N(R^{4a})(R^{4b});
R^{4a} is selected from the group consisting of optionally substituted C₁₋₆ alkyl, optionally substituted C₃₋₆ cycloalkyl, heterocyclo, heteroalkyl, (cycloalkyl)alkyl, and (heterocyclo)alkyl;
R^{4b} is selected from the group consisting of hydrogen and C₁₋₄ alkyl;
R⁵ is selected from the group consisting of optionally substituted C₁₋₆ alkyl, heterocyclo, heteroalkyl, (cycloalkyl)alkyl, and (heterocyclo)alkyl;
R^{6a}, R^{6c}, R^{6e}, R^{6f}, and R^{6g} are each independently selected from the group consisting of hydrogen, optionally substituted C₁₋₆ alkyl, optionally substituted C₃₋₆ cycloalkyl, optionally substituted aryl, optionally substituted heteroaryl, heterocyclo, heteroalkyl, (cycloalkyl)alkyl, and (heterocyclo)alkyl;
R^{6b} and R^{6d} are each independently selected from the group consisting of hydrogen, C₁₋₄ alkyl, and halogen;
R⁷ is selected from the group consisting of optionally substituted C₁₋₆ alkyl, heterocyclo, heteroalkyl, (cycloalkyl)alkyl, and (heterocyclo)alkyl; and
R⁸ is selected from the group consisting of hydrogen and halogen.

2. The pharmaceutical composition as defined in claim 1, wherein,
the compound of formula (I) is selected from the group consisting of 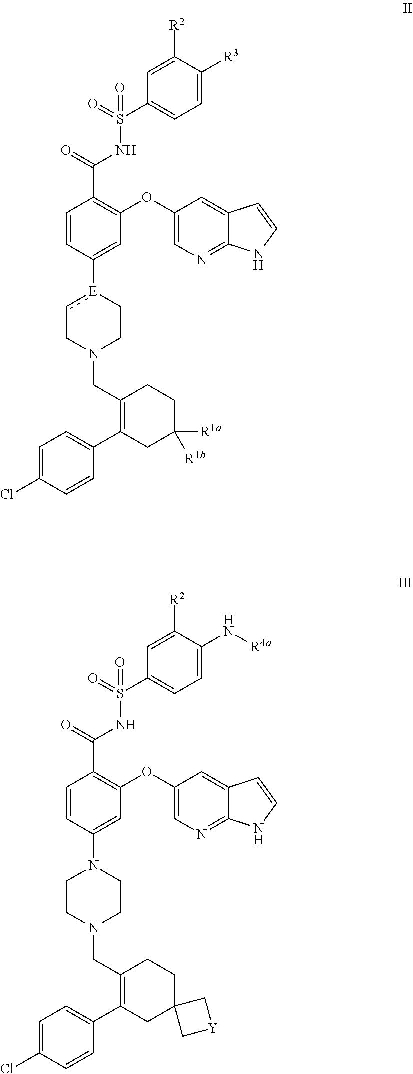 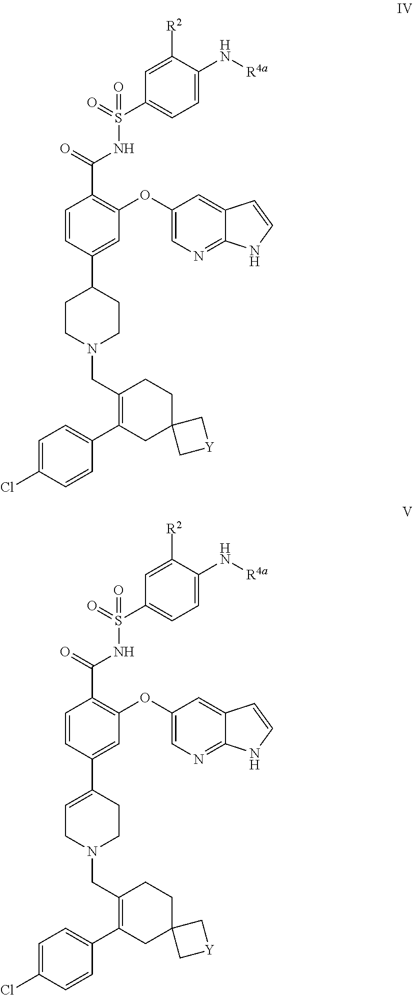 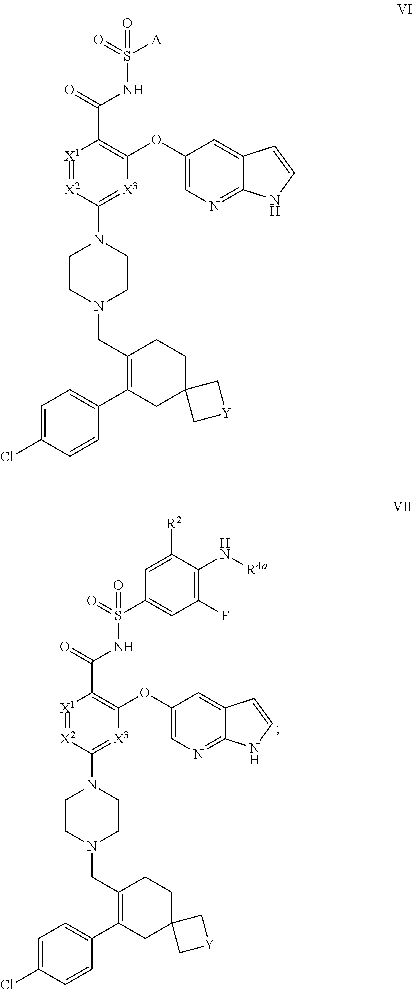
and/or, R^{4a} is selected from the group consisting of

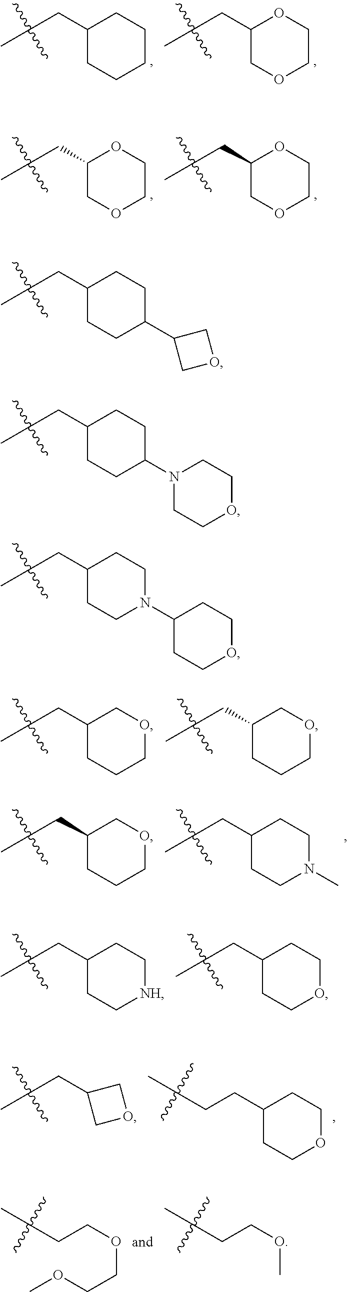

3. The pharmaceutical composition as defined in claim 1, wherein, the compound of formula (I) is selected from

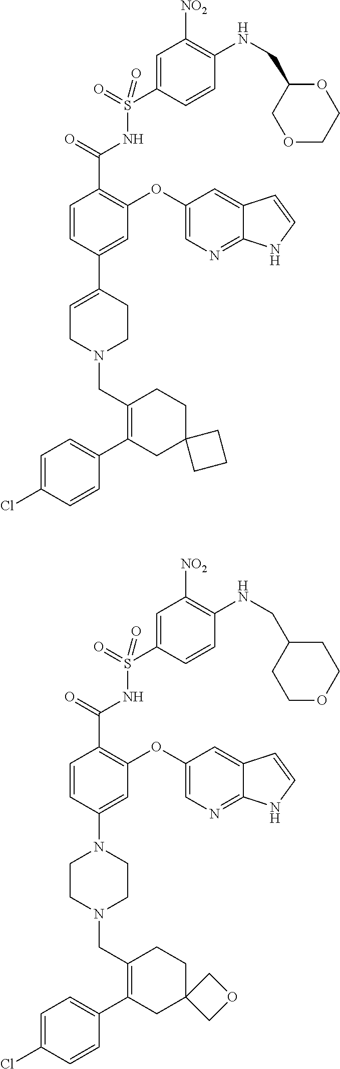 [SMILES: O=C(NS(=O)(=O)c1ccc(NC[C@@H]2COCCO2)c([N+](=O)[O-])c1)c1ccc(C2=CCN(CC3=C(c4ccc(Cl)cc4)CC4(CCC4)CC3)CC2)cc1Oc1cnc2[nH]ccc2c1 ; O=C(NS(=O)(=O)c1ccc(NCC2CCOCC2)c([N+](=O)[O-])c1)c1ccc(N2CCN(CC3=C(c4ccc(Cl)cc4)CC4(CC3)COC4)CC2)cc1Oc1cnc2[nH]ccc2c1]

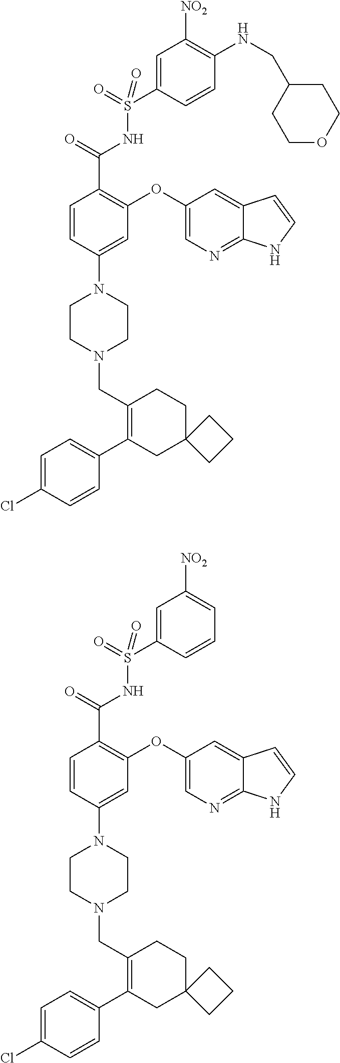 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1cccc([N+](=O)[O-])c1)c1ccc(N2CCN(CC3=C(c4ccc(Cl)cc4)CC4(CCC4)CC3)CC2)cc1Oc1cnc2[nH]ccc2c1]

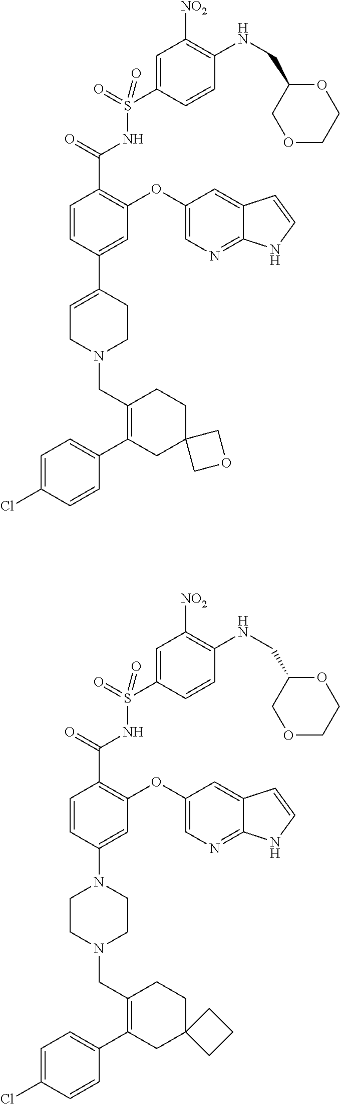 [SMILES: O=C(NS(=O)(=O)c1ccc(NC[C@@H]2COCCO2)c([N+](=O)[O-])c1)c1ccc(C2=CCN(CC3=C(c4ccc(Cl)cc4)CC4(CC3)COC4)CC2)cc1Oc1cnc2[nH]ccc2c1 ; O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

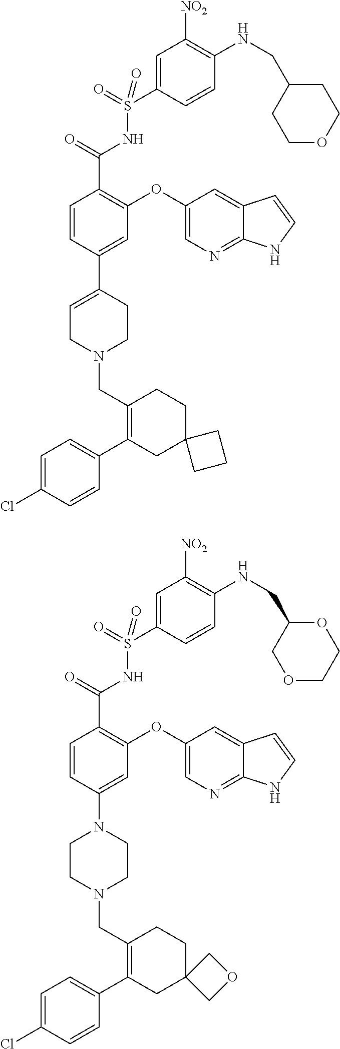 [SMILES: O=C(NS(=O)(=O)c1ccc(NCC2CCOCC2)c([N+](=O)[O-])c1)c1ccc(C2=CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@@H]2COCCO2)c([N+](=O)[O-])c1)c1ccc(N2CCN(CC3=C(c4ccc(Cl)cc4)CC4(CC3)COC4)CC2)cc1Oc1cnc2[nH]ccc2c1]

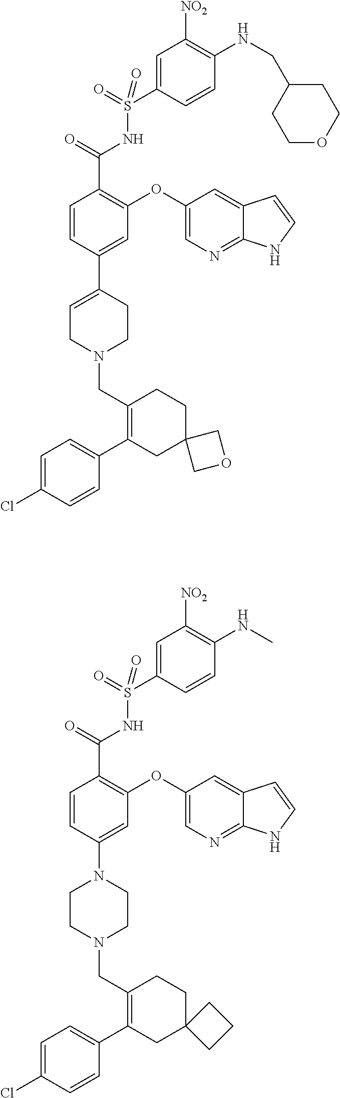 [SMILES: O=C(NS(=O)(=O)c1ccc(NCC2CCOCC2)c([N+](=O)[O-])c1)c1ccc(C2=CCN(CC3=C(c4ccc(Cl)cc4)CC4(CC3)COC4)CC2)cc1Oc1cnc2[nH]ccc2c1 ; CNc1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]]

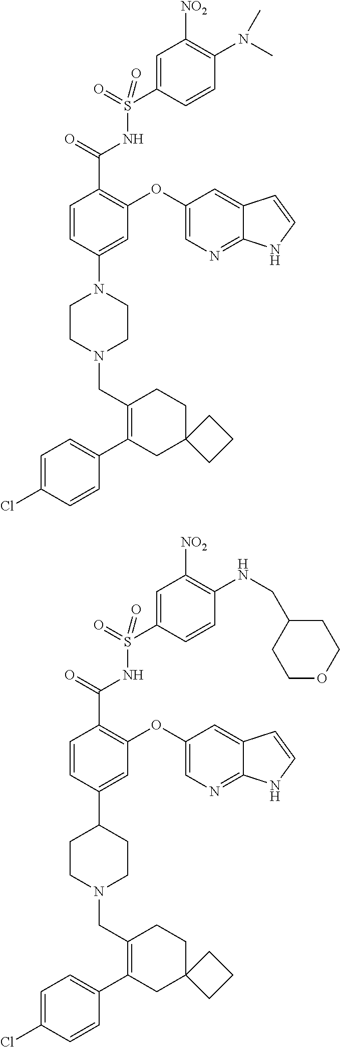 [SMILES: CN(C)c1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-] ; O=C(NS(=O)(=O)c1ccc(NCC2CCOCC2)c([N+](=O)[O-])c1)c1ccc(C2CCN(CC3=C(c4ccc(Cl)cc4)CC4(CCC4)CC3)CC2)cc1Oc1cnc2[nH]ccc2c1]

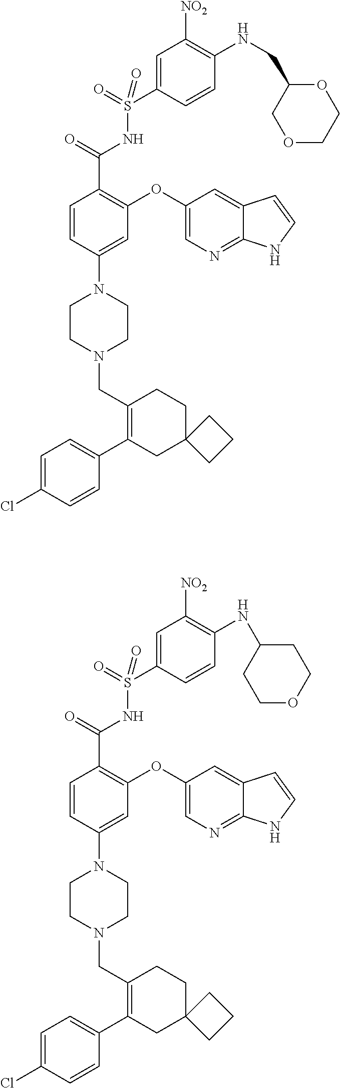 [SMILES: O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC2CCOCC2)c([N+](=O)[O-])c1)c1ccc(N2CCN(CC3=C(c4ccc(Cl)cc4)CC4(CCC4)CC3)CC2)cc1Oc1cnc2[nH]ccc2c1]

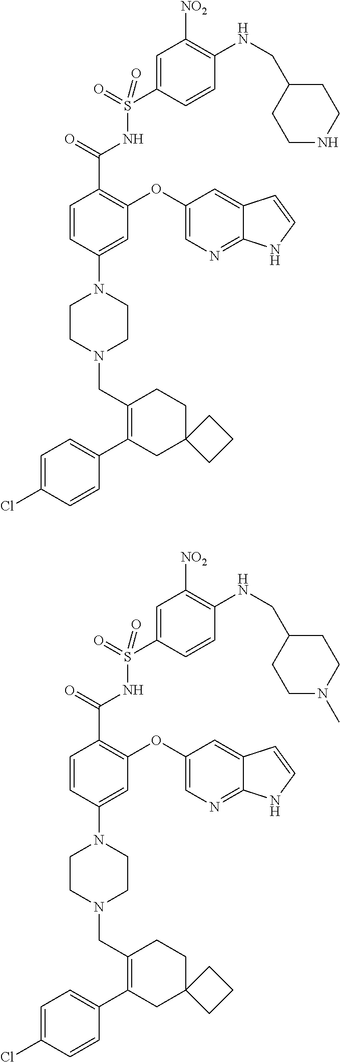 [SMILES: O=C(NS(=O)(=O)c1ccc(NCC2CCNCC2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; CN1CCC(CNc2ccc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc2[N+](=O)[O-])CC1]

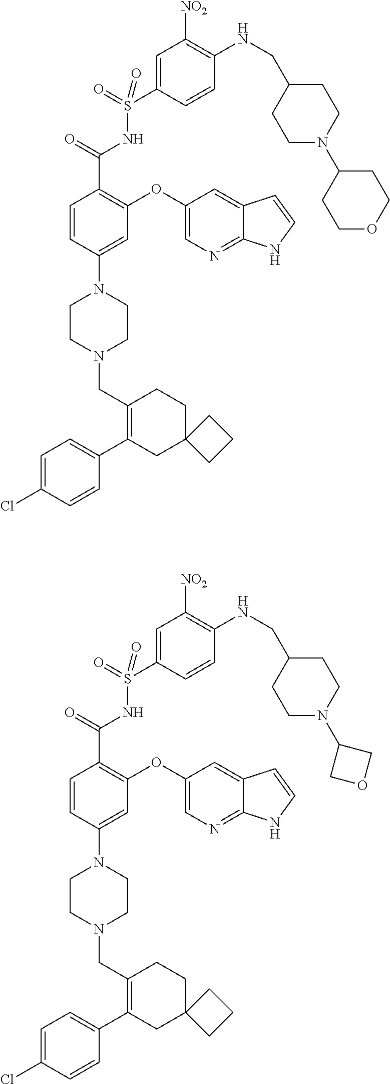 [SMILES: O=C(NS(=O)(=O)c1ccc(NCC2CCN(C3CCOCC3)CC2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NCC2CCN(C3COC3)CC2)c([N+](=O)[O-])c1)c1ccc(N2CCN(CC3=C(c4ccc(Cl)cc4)CC4(CCC4)CC3)CC2)cc1Oc1cnc2[nH]ccc2c1]

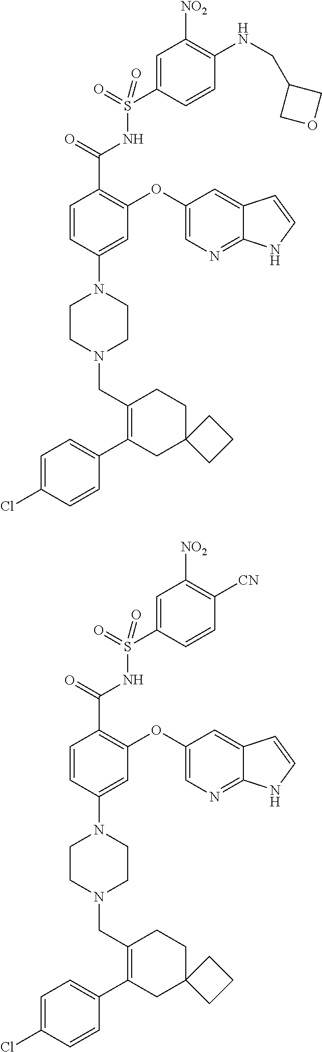 [SMILES: O=C(NS(=O)(=O)c1ccc(NCC2COC2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; N#Cc1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]]

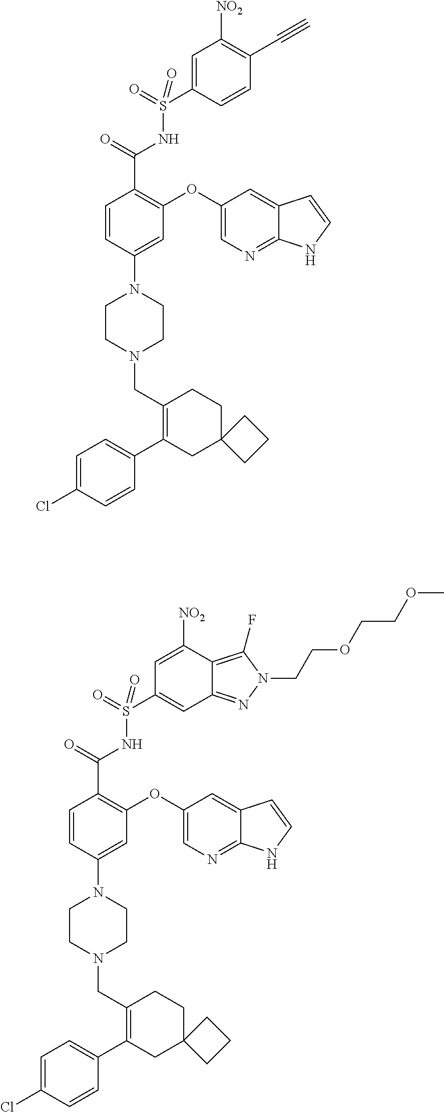 [SMILES: C#Cc1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-] ; COCCOCCn1nc2cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc([N+](=O)[O-])c2c1F]

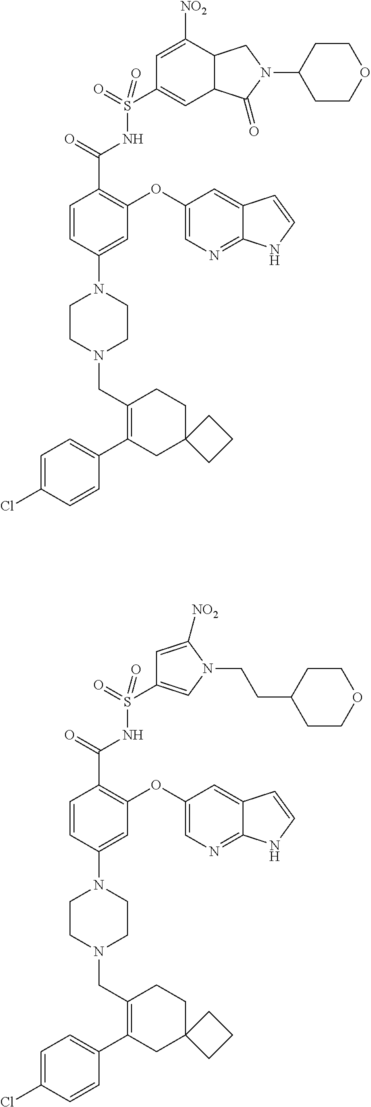 [SMILES: O=C(NS(=O)(=O)C1=CC2C(=O)N(C3CCOCC3)CC2C([N+](=O)[O-])=C1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1cc([N+](=O)[O-])n(CCC2CCOCC2)c1)c1ccc(N2CCN(CC3=C(c4ccc(Cl)cc4)CC4(CCC4)CC3)CC2)cc1Oc1cnc2[nH]ccc2c1]

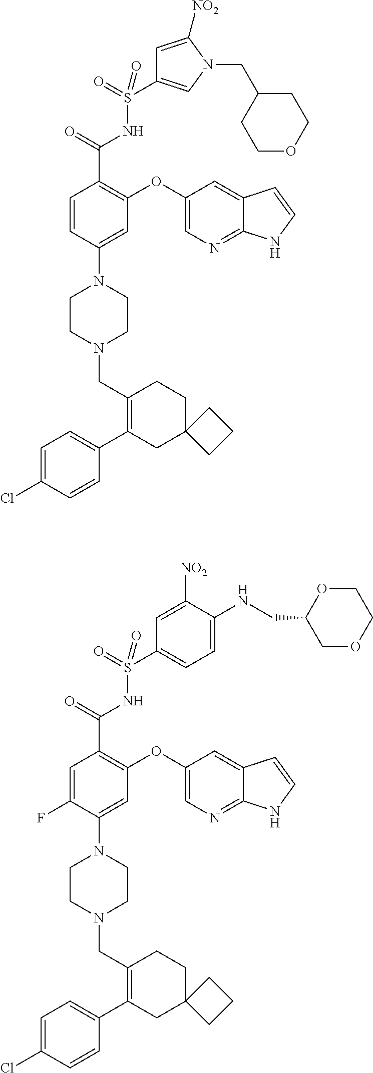 [SMILES: O=C(NS(=O)(=O)c1cc([N+](=O)[O-])n(CC2CCOCC2)c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@H]2COCCO2)c([N+](=O)[O-])c1)c1cc(F)c(N2CCN(CC3=C(c4ccc(Cl)cc4)CC4(CCC4)CC3)CC2)cc1Oc1cnc2[nH]ccc2c1]

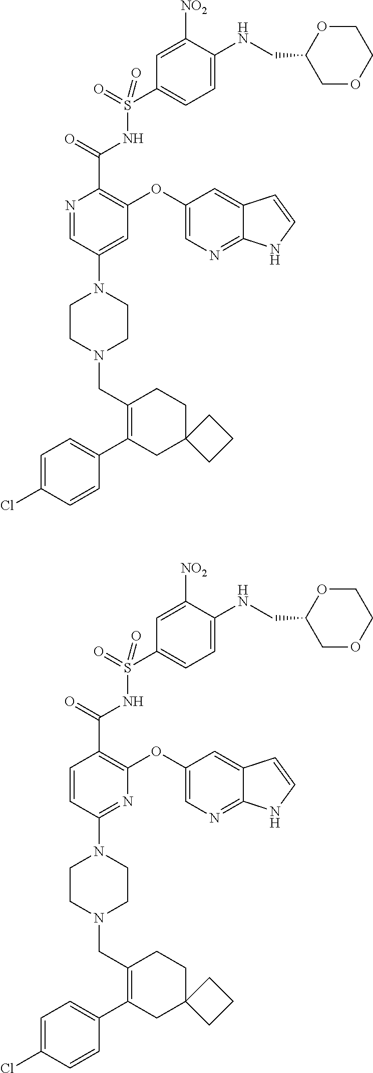 [SMILES: O=C(NS(=O)(=O)c1ccc(NC[C@H]2COCCO2)c([N+](=O)[O-])c1)c1ncc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)nc1Oc1cnc2[nH]ccc2c1]

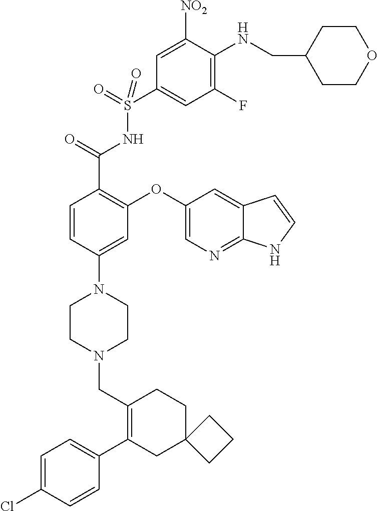 [SMILES: O=C(NS(=O)(=O)c1cc(F)c(NCC2CCOCC2)c([N+](=O)[O-])c1)c1ccc(N2CCN(CC3=C(c4ccc(Cl)cc4)CC4(CCC4)CC3)CC2)cc1Oc1cnc2[nH]ccc2c1]

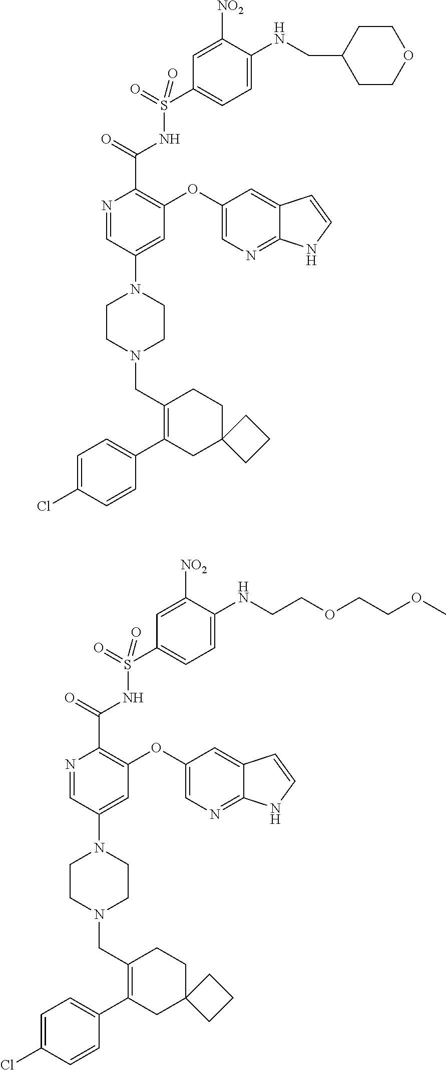 [SMILES: O=C(NS(=O)(=O)c1ccc(NCC2CCOCC2)c([N+](=O)[O-])c1)c1ncc(N2CCN(CC3=C(c4ccc(Cl)cc4)CC4(CCC4)CC3)CC2)cc1Oc1cnc2[nH]ccc2c1 ; COCCOCCNc1ccc(S(=O)(=O)NC(=O)c2ncc(N3CCN(CC4=C(c5ccc(Cl)cc5)CC5(CCC5)CC4)CC3)cc2Oc2cnc3[nH]ccc3c2)cc1[N+](=O)[O-]]

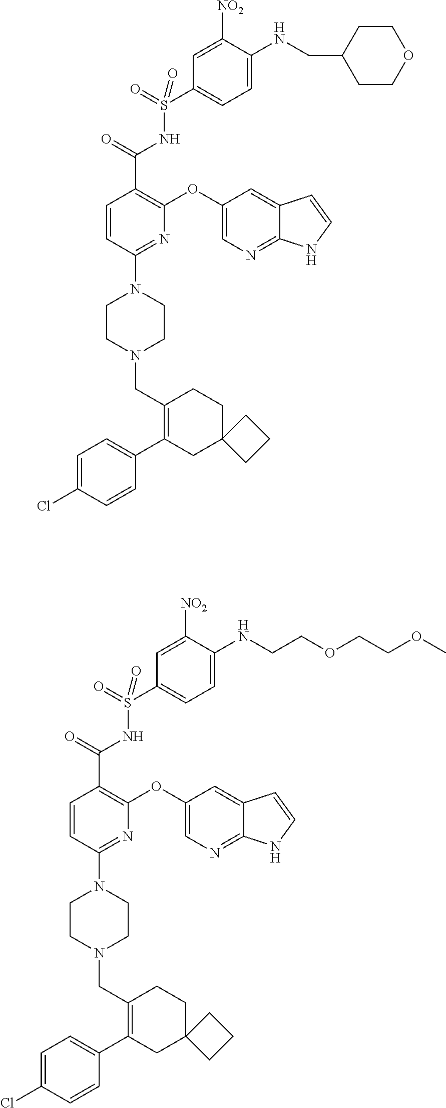 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CCC4)CC3)CC2)nc1Oc1cnc2[nH]ccc2c1 ; COCCOCCNc1ccc(S(=O)(=O)NC(=O)c2ccc(N3CCN(CC4=C(c5ccc(Cl)cc5)CC5(CCC5)CC4)CC3)nc2Oc2cnc3[nH]ccc3c2)cc1[N+](=O)[O-]]

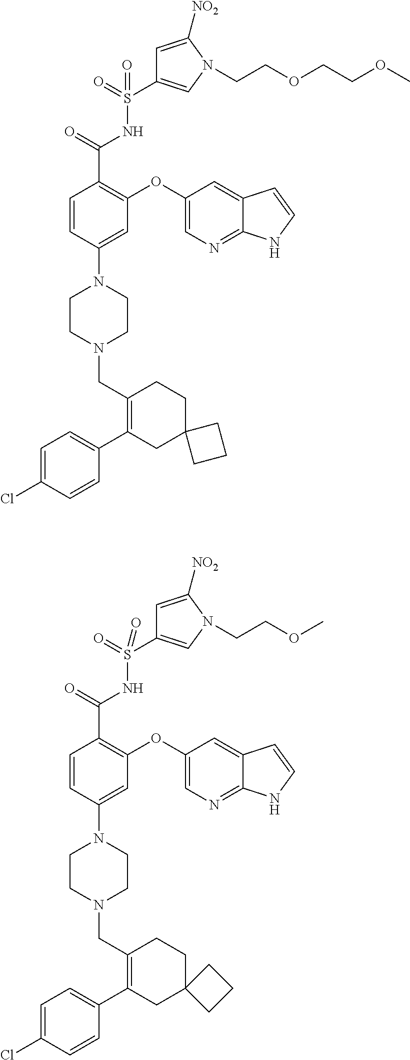 [SMILES: COCCOCCn1cc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-] ; COCCn1cc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]]

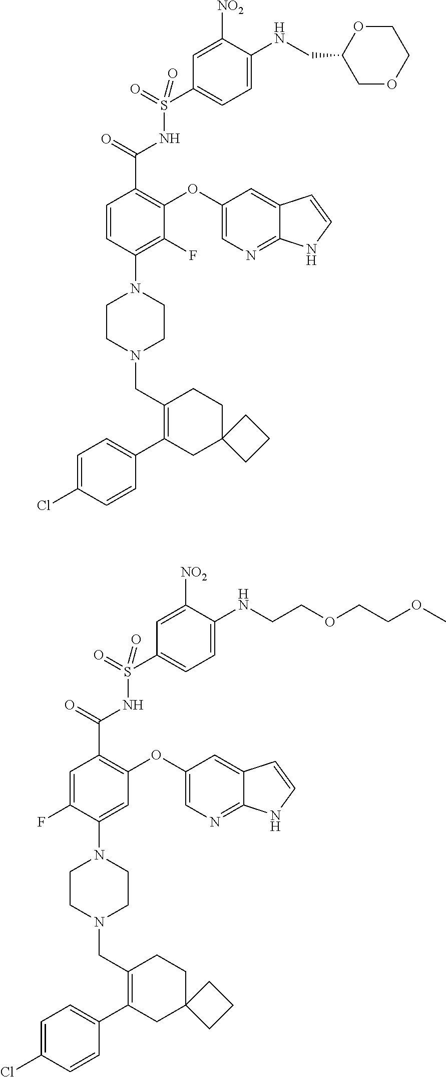 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)c(F)c1Oc1cnc2[nH]ccc2c1 ; COCCOCCNc1ccc(S(=O)(=O)NC(=O)c2cc(F)c(N3CCN(CC4=C(c5ccc(Cl)cc5)CC5(CCC5)CC4)CC3)cc2Oc2cnc3[nH]ccc3c2)cc1[N+](=O)[O-]]

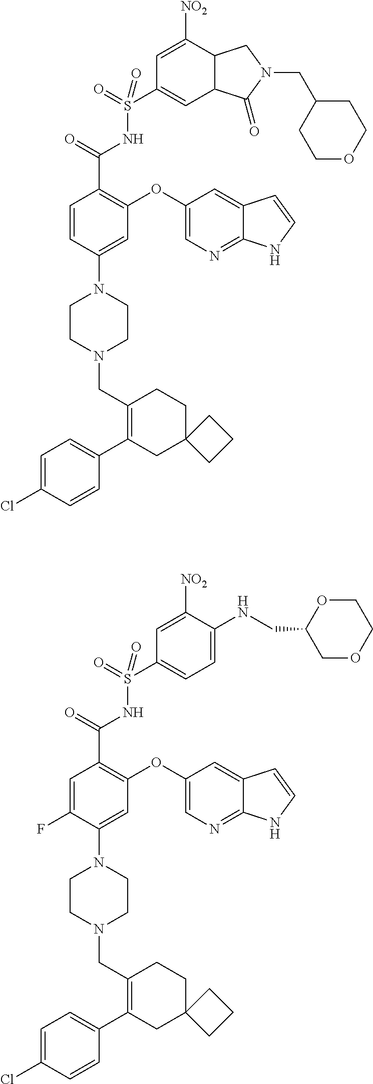 [SMILES: O=C(NS(=O)(=O)C1=CC2C(=O)N(CC3CCOCC3)CC2C([N+](=O)[O-])=C1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@H]2COCCO2)c([N+](=O)[O-])c1)c1cc(F)c(N2CCN(CC3=C(c4ccc(Cl)cc4)CC4(CCC4)CC3)CC2)cc1Oc1cnc2[nH]ccc2c1]

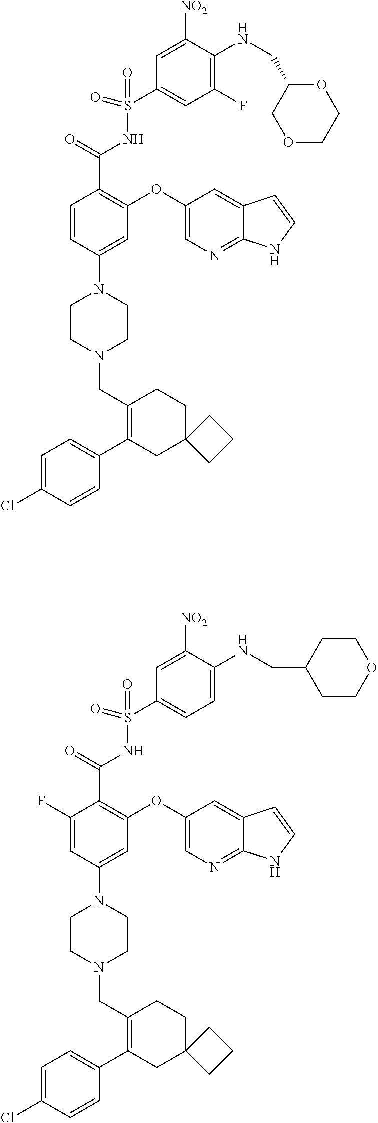 [SMILES: O=C(NS(=O)(=O)c1cc(F)c(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NCC2CCOCC2)c([N+](=O)[O-])c1)c1c(F)cc(N2CCN(CC3=C(c4ccc(Cl)cc4)CC4(CCC4)CC3)CC2)cc1Oc1cnc2[nH]ccc2c1]

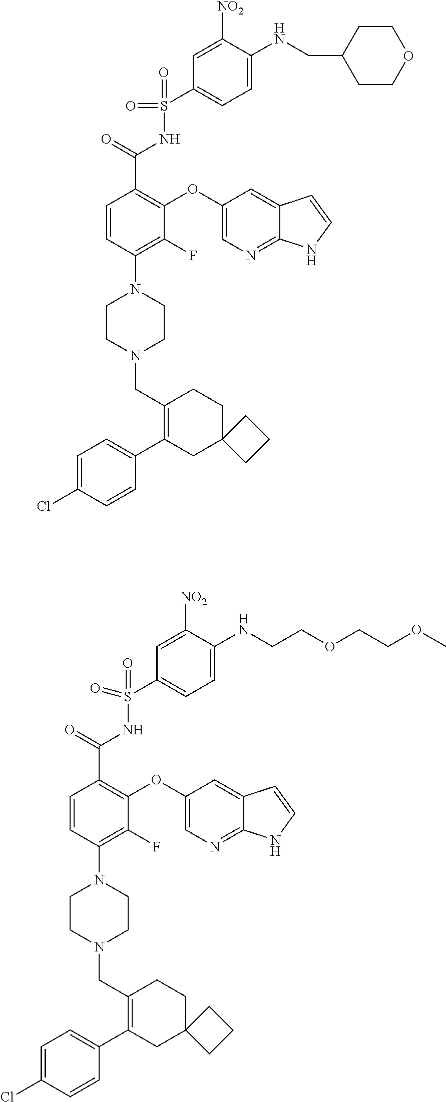 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CCC4)CC3)CC2)c(F)c1Oc1cnc2[nH]ccc2c1 ; COCCOCCNc1ccc(S(=O)(=O)NC(=O)c2ccc(N3CCN(CC4=C(c5ccc(Cl)cc5)CC5(CCC5)CC4)CC3)c(F)c2Oc2cnc3[nH]ccc3c2)cc1[N+](=O)[O-]]

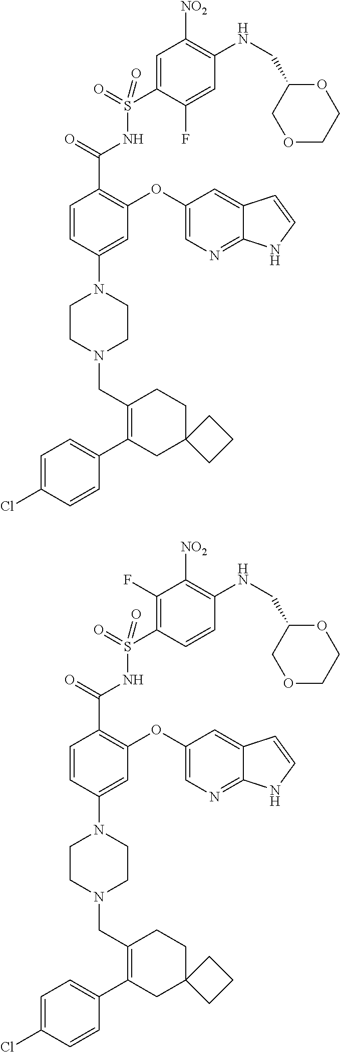 [SMILES: O=C(NS(=O)(=O)c1cc([N+](=O)[O-])c(NC[C@H]2COCCO2)cc1F)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@H]2COCCO2)c([N+](=O)[O-])c1F)c1ccc(N2CCN(CC3=C(c4ccc(Cl)cc4)CC4(CCC4)CC3)CC2)cc1Oc1cnc2[nH]ccc2c1]

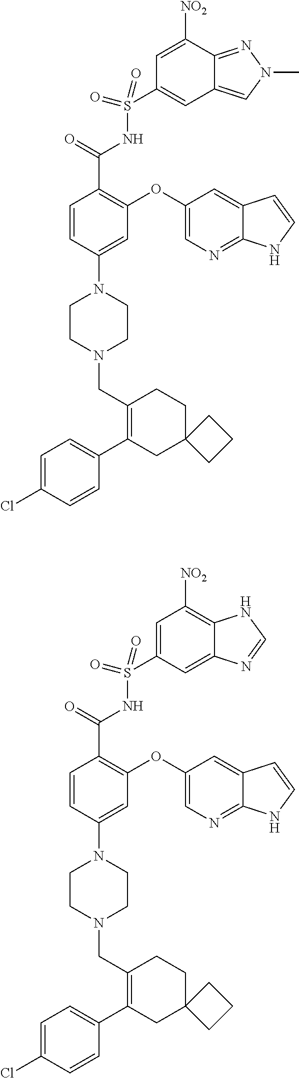 [SMILES: Cn1cc2cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc([N+](=O)[O-])c2n1 ; O=C(NS(=O)(=O)c1cc([N+](=O)[O-])c2[nH]cnc2c1)c1ccc(N2CCN(CC3=C(c4ccc(Cl)cc4)CC4(CCC4)CC3)CC2)cc1Oc1cnc2[nH]ccc2c1]

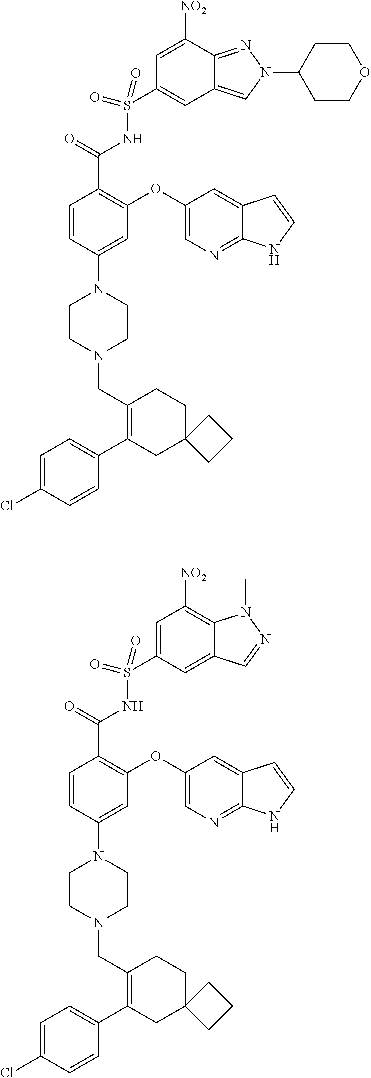 [SMILES: O=C(NS(=O)(=O)c1cc([N+](=O)[O-])c2nn(C3CCOCC3)cc2c1)c1ccc(N2CCN(CC3=C(c4ccc(Cl)cc4)CC4(CCC4)CC3)CC2)cc1Oc1cnc2[nH]ccc2c1 ; Cn1ncc2cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc([N+](=O)[O-])c21]

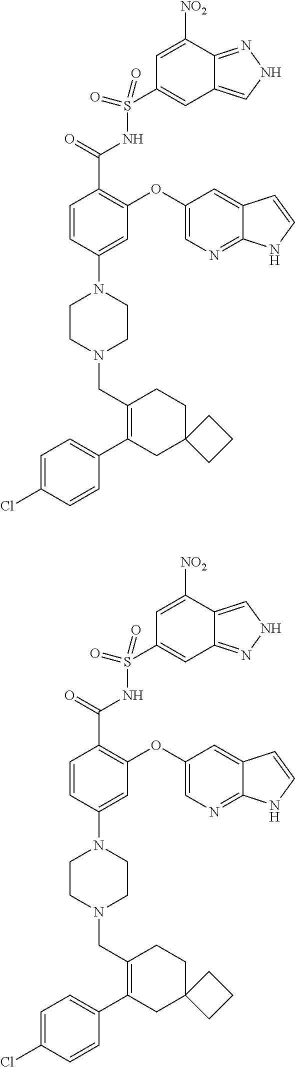 [SMILES: O=C(NS(=O)(=O)c1cc([N+](=O)[O-])c2n[nH]cc2c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1cc([N+](=O)[O-])c2c[nH]nc2c1)c1ccc(N2CCN(CC3=C(c4ccc(Cl)cc4)CC4(CCC4)CC3)CC2)cc1Oc1cnc2[nH]ccc2c1]

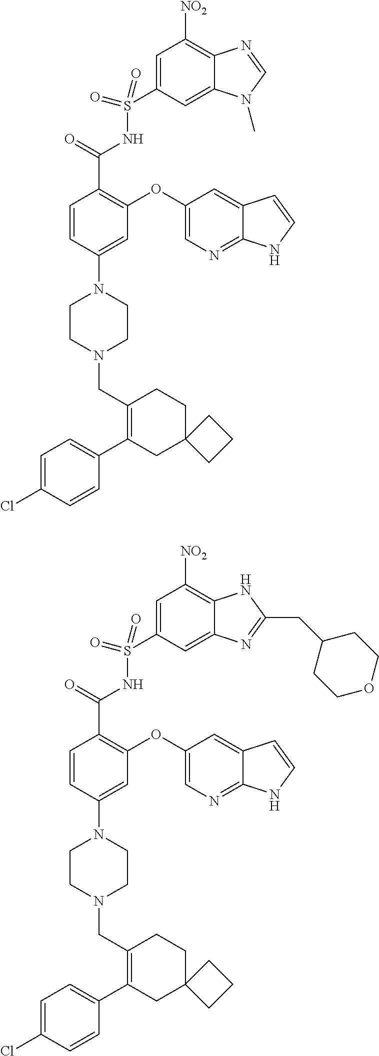 [SMILES: Cn1cnc2c([N+](=O)[O-])cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc21 ; O=C(NS(=O)(=O)c1cc([N+](=O)[O-])c2[nH]c(CC3CCOCC3)nc2c1)c1ccc(N2CCN(CC3=C(c4ccc(Cl)cc4)CC4(CCC4)CC3)CC2)cc1Oc1cnc2[nH]ccc2c1]

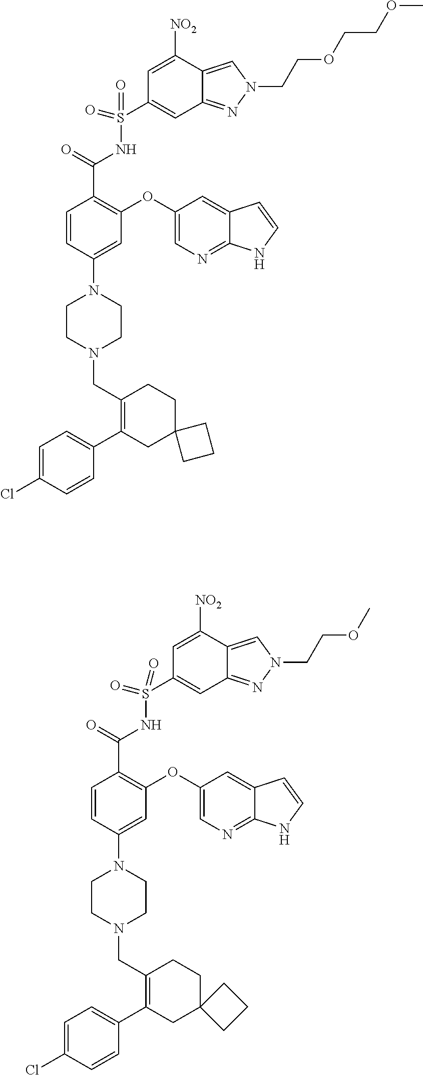 [SMILES: COCCOCCn1cc2c([N+](=O)[O-])cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc2n1 ; COCCn1cc2c([N+](=O)[O-])cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc2n1]

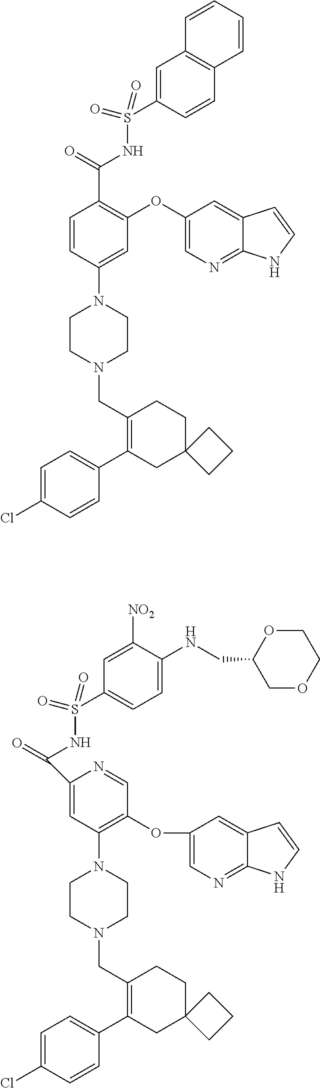 [SMILES: O=C(NS(=O)(=O)c1ccc2ccccc2c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@H]2COCCO2)c([N+](=O)[O-])c1)c1cc(N2CCN(CC3=C(c4ccc(Cl)cc4)CC4(CCC4)CC3)CC2)c(Oc2cnc3[nH]ccc3c2)cn1]

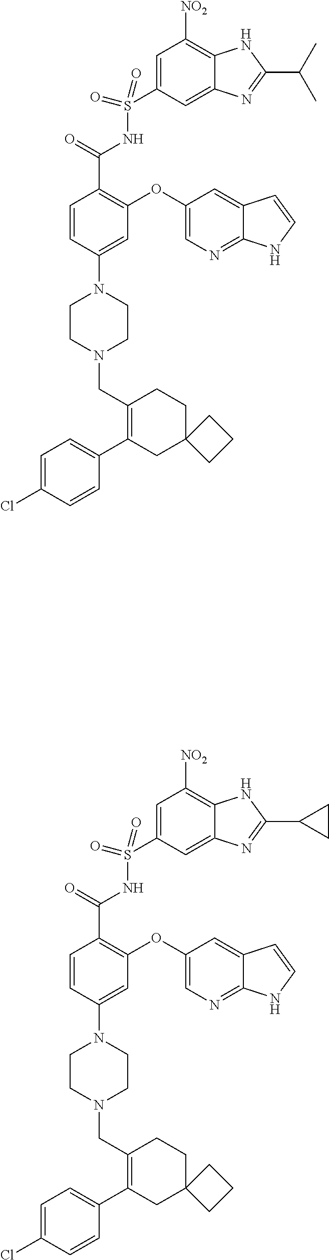 [SMILES: CC(C)c1nc2cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc([N+](=O)[O-])c2[nH]1 ; O=C(NS(=O)(=O)c1cc([N+](=O)[O-])c2[nH]c(C3CC3)nc2c1)c1ccc(N2CCN(CC3=C(c4ccc(Cl)cc4)CC4(CCC4)CC3)CC2)cc1Oc1cnc2[nH]ccc2c1]

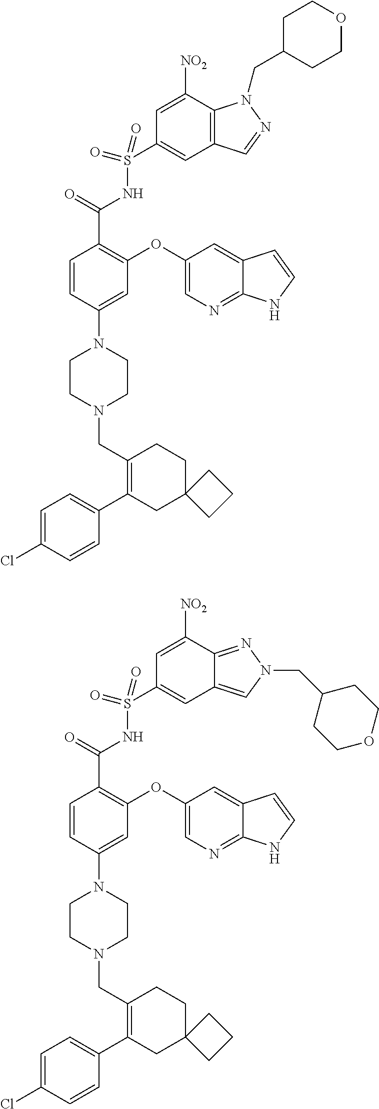 [SMILES: O=C(NS(=O)(=O)c1cc([N+](=O)[O-])c2c(cnn2CC2CCOCC2)c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1cc([N+](=O)[O-])c2nn(CC3CCOCC3)cc2c1)c1ccc(N2CCN(CC3=C(c4ccc(Cl)cc4)CC4(CCC4)CC3)CC2)cc1Oc1cnc2[nH]ccc2c1]

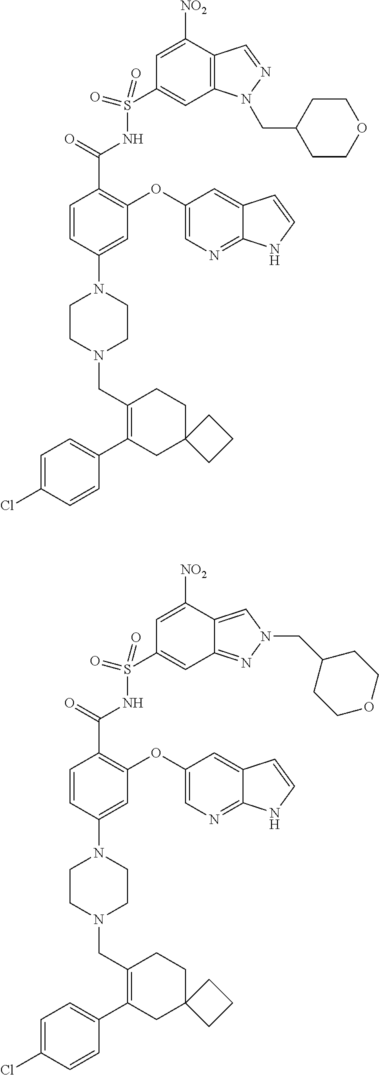 [SMILES: O=C(NS(=O)(=O)c1cc([N+](=O)[O-])c2cnn(CC3CCOCC3)c2c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1cc([N+](=O)[O-])c2cn(CC3CCOCC3)nc2c1)c1ccc(N2CCN(CC3=C(c4ccc(Cl)cc4)CC4(CCC4)CC3)CC2)cc1Oc1cnc2[nH]ccc2c1]

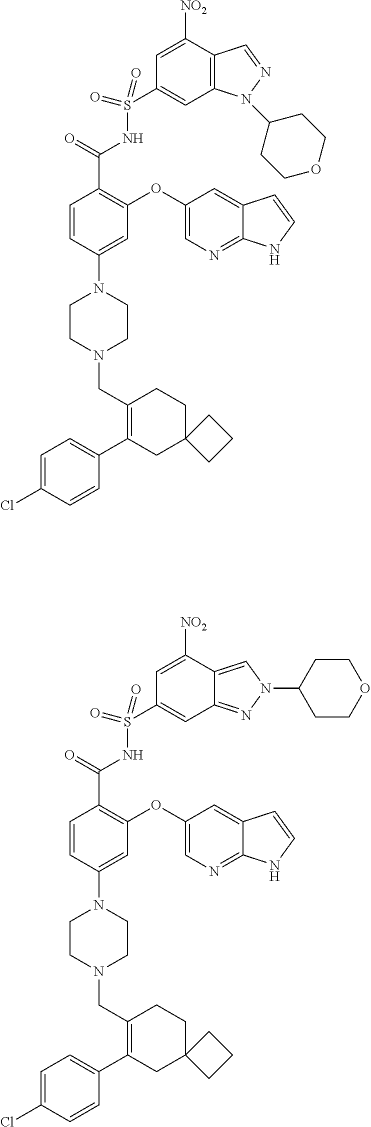 [SMILES: O=C(NS(=O)(=O)c1cc([N+](=O)[O-])c2cnn(C3CCOCC3)c2c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1cc([N+](=O)[O-])c2cn(C3CCOCC3)nc2c1)c1ccc(N2CCN(CC3=C(c4ccc(Cl)cc4)CC4(CCC4)CC3)CC2)cc1Oc1cnc2[nH]ccc2c1]

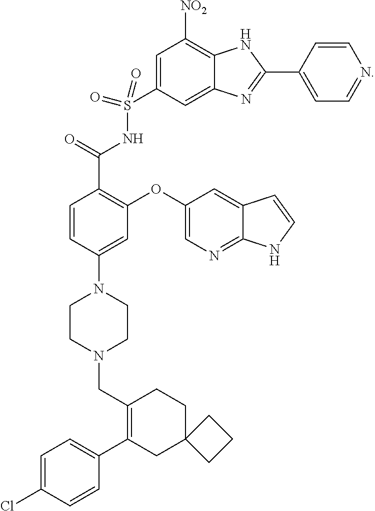 [SMILES: O=C(NS(=O)(=O)c1cc([N+](=O)[O-])c2[nH]c(-c3ccncc3)nc2c1)c1ccc(N2CCN(CC3=C(c4ccc(Cl)cc4)CC4(CCC4)CC3)CC2)cc1Oc1cnc2[nH]ccc2c1]

4. The pharmaceutical composition as defined in claim 1, wherein, the compound of formula (I) is (R)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide, (S)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide, (S)—N-((4-(((1,4-dioxan-2-yl)methyl)amino)-3-fluoro-5-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide, or a pharmaceutically acceptable salt thereof.

5. The pharmaceutical composition as defined in claim 1, wherein, the compound of formula (I) is (S)—N-((4-(((1,4-dioxan-2-yl)methyl)amino)-3-fluoro-5-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide.

6. The pharmaceutical composition as defined in claim 1, wherein, the CDK inhibitor is selected from the group consisting of kenpaullone, PKC-412, butyrolactone I, alvocidib, N9-isopropyl-olomoucine, indirubin-3′-monoxime, NU2058, olomoucine II, 9-cyanopaullone, 5-iodo-indirubin-3′-monoxime, NU6102, oxindole I, SU 9516, roscovitine, RO-3306, 10Z-hymenialdisine, AZD 5438, AT7519, dinaciclib, R547, CGP 74514A, SNS-032, BMS-265246, JNJ-7706621, PHA-793887, P276-00, PHA-767491, milciclib, NU6027, LDC000067, ribociclib, palbociclib, abemaciclib, Senexin A, Atuveciclib, LY2857785, and dinaciclib.

7. The pharmaceutical composition as defined in claim 1, wherein, the CDK inhibitor is a CDK9 inhibitor or a pharmaceutically acceptable salt thereof.

8. The pharmaceutical composition as defined in claim 1, wherein, the CDK inhibitor is compound 2 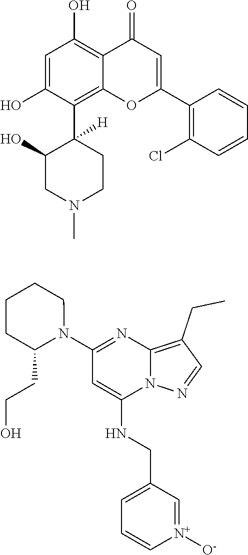 [SMILES: CN1CC[C@H](c2c(O)cc(O)c3c(=O)cc(-c4ccccc4Cl)oc23)[C@H](O)C1 ; CCc1cnn2c(NCc3ccc[n+]([O-])c3)cc(N3CCCC[C@H]3CCO)nc12]
Compound 2, compound 3 Compound 3, or a pharmaceutically acceptable salt thereof.

9. The pharmaceutical composition as defined in claim 1, wherein, the compound of formula (I) is (R)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide, (S)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide, (S)—N-((4-(((1,4-dioxan-2-yl)methyl)amino)-3-fluoro-5-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide, or a pharmaceutically acceptable salt thereof;
and, the CDK inhibitor is compound 2 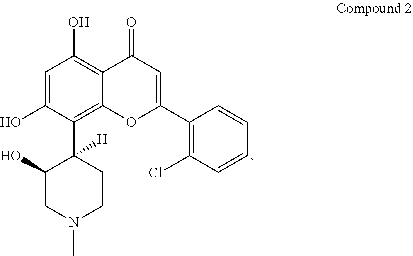 [SMILES: CN1CC[C@H](c2c(O)cc(O)c3c(=O)cc(-c4ccccc4Cl)oc23)[C@H](O)C1]
compound 3 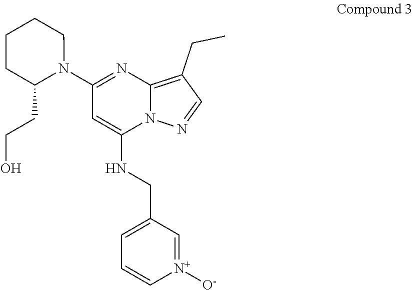 [SMILES: CCc1cnn2c(NCc3ccc[n+]([O-])c3)cc(N3CCCC[C@H]3CCO)nc12]
or a pharmaceutically acceptable salt thereof.

10. The pharmaceutical composition as defined in claim 1, wherein, the compound of formula (I) is (S)—N-((4-(((1,4-dioxan-2-yl)methyl)amino)-3-fluoro-5-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide or a pharmaceutically acceptable salt thereof, and the CDK inhibitor is 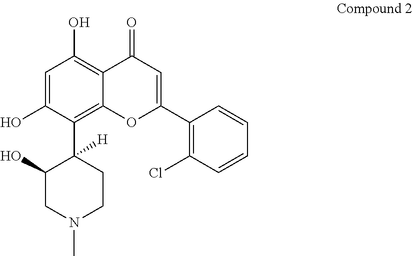 [SMILES: CN1CC[C@H](c2c(O)cc(O)c3c(=O)cc(-c4ccccc4Cl)oc23)[C@H](O)C1]
or a pharmaceutically acceptable salt thereof.

11. The pharmaceutical composition as defined in claim 1, wherein, the weight ratio of the compound of formula (I) to the CDK inhibitor is 50:1 to 1:50.

12. A pharmaceutical combination comprising
(i) a compound of formula (I) or a pharmaceutically acceptable salt thereof, and
(ii) a CDK inhibitor or a pharmaceutically acceptable salt thereof,
wherein, the compound of formula (I) and the CDK inhibitor are defined as in claim 1.

13. (canceled)

14. A method for the prevention and/or treatment of a disease mediated by Bcl-2 and/or CDK activity, which comprises administering to a subject in need thereof a therapeutically effective amount of a compound of formula (I) or a pharmaceutically acceptable salt thereof, and a therapeutically effective amount of a CDK inhibitor or a pharmaceutically acceptable salt thereof;
wherein, the compound of formula (I) and the CDK inhibitor are defined as claim 1.

15. The method as defined in claim 14, wherein, the disease mediated by Bcl-2 and/or CDK activity is a cancer, the cancer can be selected from the group consisting of adrenal cortical cancer, advanced cancer, anal cancer, aplastic anemia, bile duct cancer, bladder cancer, bone cancer, bone metastasis, brain/CNS tumors in adults, brain/CNS tumors in children, breast cancer, breast cancer in men, cancer in children, cancer of unknown primary, Castleman disease, cervical cancer, colon/rectum cancer, endometrial cancer, esophagus cancer, Ewing family of tumors, eye cancer, gallbladder cancer, gastrointestinal carcinoid tumors, gastrointestinal stromal tumor, gestational trophoblastic disease, Hodgkin disease, Kaposi sarcoma, kidney cancer, laryngeal and hypopharyngeal cancer, leukemia, liver cancer, lung cancer-non-small cell, lung cancer-small cell, lung carcinoid tumor, lymphoma of the skin, malignant mesothelioma, myelodysplastic syndrome, nasal cavity and paranasal sinus cancer, nasopharyngeal cancer, neuroblastoma, neuroectodermal tumor, peritoneal cancer, human head and neck squamous cell carcinoma, non-Hodgkin lymphoma, non-Hodgkin lymphoma in children, Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma-adult soft tissue cancer, skin cancer-basal and squamous cell, skin cancer-melanoma, small intestine cancer, stomach cancer, testicular cancer, thymus cancer, thyroid cancer, uterine sarcoma, vaginal cancer, vulvar cancer, Waldenstrom macroglobulinemia, and Wilms Tumor;
or, the disease mediated by Bcl-2 and/or CDK activity is cardiac hypertrophy, dilated cardiomyopathy, atherosclerosis, muscle atrophy or obesity.

16. The method as defined in claim 14, wherein, the disease mediated by Bcl-2 and/or CDK activity is non-Hodgkin lymphoma or leukemia, the non-Hodgkin lymphoma is preferably diffuse large B-cell lymphoma; the leukemia is preferably myelodysplastic syndrome, chronic lymphocytic leukemia or acute myeloid leukemia.

17. The method as defined in claim 14, wherein, the compound of formula (I) and the CDK inhibitor are administered simultaneously or separately in any order;
and/or, the compound of formula (I) and the CDK inhibitor are administered to a subject in a weight ratio of 50:1 to 1:50.

18. (canceled)

19. A method for the prevention and/or treatment of a cancer, which comprises administering to a subject in need thereof a therapeutically effective amount of a compound of formula (I) or a pharmaceutically acceptable salt thereof, and a therapeutically effective amount of a CDK inhibitor or a pharmaceutically acceptable salt thereof;
wherein, the compound of formula (I) and the CDK inhibitor are defined as claim 1.

20. The method as defined in claim 19, wherein, the cancer is selected from the group consisting of adrenal cortical cancer, advanced cancer, anal cancer, aplastic anemia, bile duct cancer, bladder cancer, bone cancer, bone metastasis, brain/CNS tumors in adults, brain/CNS tumors in children, breast cancer, breast cancer in men, cancer in children, cancer of unknown primary, Castleman disease, cervical cancer, colon/rectum cancer, endometrial cancer, esophagus cancer, Ewing family of tumors, eye cancer, gallbladder cancer, gastrointestinal carcinoid tumors, gastrointestinal stromal tumor, gestational trophoblastic disease, Hodgkin disease, Kaposi sarcoma, kidney cancer, laryngeal and hypopharyngeal cancer, leukemia, liver cancer, lung cancer-non-small cell, lung cancer-small cell, lung carcinoid tumor, lymphoma of the skin, malignant mesothelioma, myelodysplastic syndrome, nasal cavity and paranasal sinus cancer, nasopharyngeal cancer, neuroblastoma, neuroectodermal tumor, peritoneal cancer, human head and neck squamous cell carcinoma, non-Hodgkin lymphoma, non-Hodgkin lymphoma in children, Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma-adult soft tissue cancer, skin cancer-basal and squamous cell, skin cancer-melanoma, small intestine cancer, stomach cancer, testicular cancer, thymus cancer, thyroid cancer, uterine sarcoma, vaginal cancer, vulvar cancer, Waldenstrom macroglobulinemia, and Wilms Tumor.

21. The method as defined in claim 19, wherein, the cancer is non-Hodgkin lymphoma or leukemia, the non-Hodgkin lymphoma is preferably diffuse large B-cell lymphoma; the leukemia is preferably myelodysplastic syndrome, chronic lymphocytic leukemia or acute myeloid leukemia.

22. A kit comprising in separate containers in a single package pharmaceutical compositions comprising in one container a pharmaceutical composition comprising a compound of formula (I) or a pharmaceutically acceptable salt thereof, and in a second container a pharmaceutical composition comprising a CDK inhibitor or a pharmaceutically acceptable salt thereof,
wherein, the compound of formula (I) and the CDK inhibitor are defined as in claim 1.
